(19) United States
(12) Patent Application Publication
(10) Pub. No.: US 2009/0062235 A1
(43) Pub. Date: Mar. 5, 2009

(54) Inhibitors of Dipeptidylpeptidase IV

(75) Inventors: William W. Bachovchin, Cambridge, MA (US); Hung-Sen Lai, Andover, MA (US); Wengen Wu, Meford, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Trustees of Tufts College, Boston, MA (US)

(21) Appl. No.: 12/263,679

(22) Filed: Nov. 3, 2008

Related U.S. Application Data
(62) Division of application No. 11/065,001, filed on Feb. 23, 2005.
(60) Provisional application No. 60/547,227, filed on Feb. 23, 2004.
(60) Provisional application No. 60/599,336, filed on Aug. 6, 2004.

(51) Int. Cl. A61K 31/69; C07F 5/02

(52) U.S. Cl. 514/64; 562/7

(57) ABSTRACT

The present invention relates to inhibitors of post-proline cleaving enzymes, such as inhibitors of dipeptidyl peptidase IV, as well as pharmaceutical compositions thereof, and methods for using such inhibitors. In particular, the inhibitors of the present invention are improved over those in the prior art by selection of particular classes of sidechains in the P1 and/or P2 position of the inhibitor that contain a carboxylic acid moiety. The compounds of the present invention can have a better therapeutic index, owing in part to reduced toxicity and/or improved specificity for the targeted protease.

### RELATED APPLICATIONS

This application claims the benefit of U.S. Provisional Application Nos. 60/547,227, filed Feb. 23, 2004 and 60/599,336, filed Aug. 6, 2004. The teachings of these applications are incorporated herein by reference in their entirety.

### BACKGROUND OF THE INVENTION

Proteases are enzymes that cleave proteins at single, specific peptide bonds. Proteases can be classified into four generic classes: serine, thiol or cysteinyl, acid or aspartyl, and metalloproteases (Cuypers et al., J. Biol. Chem. 257:7086 (1982)). Proteases are essential to a variety of biological activities, such as digestion, formation, and dissolution of blood clots, reproduction, and the immune reaction to foreign cells and organisms. Aberrant proteolysis is associated with a number of disease states in man and other mammals. In many instances, it is beneficial to disrupt the function of one or more proteolytic enzymes in the course of therapeutically treating an animal.

The binding site for a peptide substrate consists of a series of “specificity subsites” across the surface of the enzyme. The term “specificity subsite” refers to a pocket or other site on the enzyme capable of interacting with a portion of a substrate for the enzyme. In discussing the interactions of peptides with proteases, e.g., serine and cysteine proteinases, and the like, the present application utilizes the nomenclature of Schechter and Berger [(1967) Biochem. Biophys. Res. Commun. 27:157-162)]. The individual amino acid residues of a substrate or inhibitor are designated P1, P2, etc. and the corresponding subsites of the enzyme are designated S1, S2, etc, starting with the carboxy terminal residue produced in the cleavage reaction. The scissile bond of the substrate is the amide bond between P1-P1′ of the substrate. Thus, for a peptide Xaa1-Xaa2-Xaa3-Xaa4 which is cleaved between the Xaa3 and Xaa4 residues, the Xaa3 residue is referred to as the P1 residue and binds to the S1 subsite of the enzyme, Xaa2 is referred to as the P2 residue and binds to the S2 subsite, and so forth.

Dipeptidyl peptidase IV (DPIV), for example, is a serine protease which cleaves N-terminal dipeptides from a peptide chain containing, preferably, a proline residue in the penultimate position, e.g., in the P1 position. DPIV belongs to a group of cell-membrane-associated peptidases and, like the majority of cell-surface peptidases, is a type II integral membrane protein, being anchored to the plasma membrane by its signal sequence. DPIV is found in a variety of differentiated mammalian epithelia, endothelia and hematopoetic cells and tissues, including those of lymphoid origin where it is found specifically on the surface of CD4⁺ T cells. DPIV has been identified as the leukocyte differentiation marker CD26.

### SUMMARY OF THE INVENTION

One aspect of the invention provides a protease inhibitor having a structure of Formula I

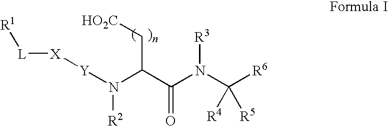

or a pharmaceutically acceptable salt thereof, where:

R¹ represents H, alkyl, alkoxy, alkenyl, alkynyl, amino, alkylamino, acylamino, cyano, sulfonylamino, acyloxy, aryl, cycloalkyl, heterocyclyl, heteroaryl, or a polypeptide chain of 1 to 8 amino acid residues;

R² represents H, lower alkyl, or aralkyl;

R³ and R⁴ independently represent H, halogen, or alkyl, or R³ and R⁴ together with the carbon to which they are attached, form a 3- to 6-membered heterocyclic ring;

R⁵ represents H, halogen, lower alkyl, or aralkyl, preferably H or lower alkyl;

R⁶ represents a functional group that reacts with an active site residue of a targeted protease to form a covalent adduct;

R⁷ represents H, aryl, alkyl, aralkyl, cycloalkyl, heterocyclyl, heteroaryl, heteroaralkyl, or a polypeptide chain of 1 to 8 amino acid residues;

L is absent or represents alkyl, alkenyl, alkynyl, —(CH₂)ₘ—O—(CH₂)ₘ—, —(CH₂)ₘNR₂(CH₂)ₘ—, and —(CH₂)ₘS(CH₂)ₘ—;

X is absent or represents —N(R⁷)—, —O—, or —S—;

Y is absent or represents —C(═O)—, —C(═S)—, or —SO₂—;

m is, independently for each occurrence, an integer from 0 to 10, preferably from 1 to 3; and

n is an integer from 1 to 6.

In certain preferred embodiments, R¹ represents H or lower alkyl, R³ is H and R⁴ is lower alkyl, or R³ and R⁴ together with the carbon to which they are attached form a 5-membered ring, and n is 2.

In certain other preferred embodiments R¹ represents H or lower alkyl, R³ represents H, R⁴ represents H or lower alkyl, R⁵ represents H, and n is 2.

In certain preferred embodiments where X, Y, and L are absent, R¹ is a polypeptide chain of 2 to 8 amino acid residues, where proline is the residue that is directly attached to the leftmost residue of Formula I. In certain such embodiments, R¹ is a polypeptide chain of 2 amino acid residues, where proline is the residue that is directly attached to the leftmost nitrogen of Formula I.

In certain of the above embodiments, R⁶ represents boronic acid, CN, —SO₂Z¹, —P(═O)Z¹, —P(═R⁸)R⁹R¹⁰, —C(═NH)NH₂, —CH═NR¹¹, or —C(═O)—R¹¹ where:

R⁸ is O or S;

R⁹ represents N₃, SH₂, NH₂, NO₂, or OLR¹², and

R¹⁰ represents lower alkyl, amino, OLR¹², or a pharmaceutically acceptable salt thereof, or

R⁹ and R¹⁰, together with the phosphorus to which they are attached, form a 5- to 8-membered heterocyclic ring;

R¹¹ represents H, alkyl, alkenyl, alkynyl, NH₂, —(CH₂)ₚ—R¹², —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)NH₂, —C(O)OR¹³, or —C(Z¹)(Z²)(Z³);

R¹² represents H, alkyl, alkenyl, aryl, heteroaryl, cycloalkyl, cycloalkenyl, or heterocyclyl;

R¹³ represents H, alkyl, alkenyl, or LR¹²;

Z¹ represents a halogen;

Z² and Z³ independently represent H or halogen;

p is, independently for each occurrence, an integer from 0 to 8; and

q is, independently for each occurrence, an integer from 1 to 8.

In certain preferred embodiments, R⁶ represents CN, CHO, or C(═O)C(Z¹)(Z²)(Z³), where Z¹ represents a halogen, and Z² and Z³ represent H or halogen. In another embodiment, R⁶ represents C(═O)C(Z¹)(Z²)(Z³), where Z¹ represents fluorine, and Z² and Z³ represent H or fluorine.

In certain preferred embodiments, R⁶ represents a group of formula —B(Y¹)(Y²), where Y¹ and Y² are independently OH or a group that is hydrolysable to OH (i.e., to form a boronic acid), or together with the boron atom to which they are attached form a 5- to 8-membered ring that is hydrolysable to a boronic acid.

Another aspect of the invention relates to a protease inhibitor having a structure of Formula II:

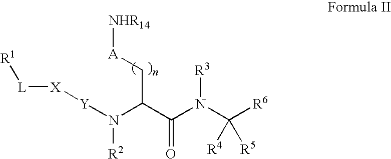

or a pharmaceutically acceptable salt thereof, where:

R¹ represents H, alkyl, alkoxy, alkenyl, alkynyl, amino, alkylamino, acylamino, cyano, sulfonylamino, acyloxy, aryl, cycloalkyl, heterocyclyl, heteroaryl, or a polypeptide chain of 1 to 8 amino acid residues;

R² represents H, lower alkyl, or aralkyl;

R³ and R⁴ independently represent H, halogen, or alkyl, or R³ and R⁴ together with the carbon to which they are attached, form a 3- to 6-membered heterocyclic ring;

R⁵ represents H, halogen, lower alkyl, or aralkyl, preferably H or lower alkyl;

R⁶ represents a functional group that reacts with an active site residue of a targeted protease to form a covalent adduct;

R⁷ represents H, aryl, alkyl, aralkyl, cycloalkyl, heterocyclyl, heteroaryl, heteroaralkyl, or polypeptide chains of 1 to 8 amino acid residues;

R¹⁴ represents H, alkyl, alkoxy, alkenyl, alkynyl, or aralkyl, preferably H;

A is absent or represents —NHC(═NH)—, or R¹⁴ and A together with the nitrogen to which they are attached form heterocyclic ring;

L is absent or represents alkyl, alkenyl, alkynyl, —(CH₂)ₘO(CH₂)ₘ—, —(CH₂)ₘNR₂(CH₂)ₘ—, or —(CH₂)ₘS(CH₂)ₘ—;

X is absent or represents —N(R⁷)—, —O—, or —S—;

Y is absent or represents —C(═O)—, —C(═S)—, or —SO₂—;

m is, independently for each occurrence, an integer from 0 to 10; and

n is an integer from 1 to 6.

In certain preferred embodiments, R¹ represents H or lower alkyl, R³ is H and R⁴ is lower alkyl, or R³ and R⁴ together with the carbon to which they are attached form a 5-membered ring, and n is an integer from 1 to 4.

In certain other preferred embodiments R¹ represents H or lower alkyl, R³ represents H, R⁴ represents H or lower alkyl, R⁵ represents H, and n is an integer from 1 to 4.

In certain preferred embodiments where X, Y, and L are absent, R¹ is a polypeptide chain of 2 to 8 amino acid residues, where proline is the residue that is directly attached to the leftmost residue of Formula II. In certain such embodiments, R¹ is a polypeptide chain of 2 amino acid residues, wherein proline is the residue that is directly attached to the leftmost nitrogen of Formula II.

In certain embodiments, R¹⁴ is H Or alkyl. In certain such embodiments, A is absent or is —NHC(═NH)—.

In certain preferred embodiments, R¹⁴ is H, A is absent, and n is 4. In certain other embodiments, R¹⁴ is H, A is —NHC(═NH)—, and n is 3.

In certain preferred embodiments, A and R¹⁴ together with the nitrogen to which they are attached form an imidazole ring, and n is 1.

In certain embodiments, R⁶ represents boronic acid, —CN, —SO₂Z¹, —P(═O)Z¹, —P(═R⁸)R⁹R¹⁰, —C(═NH)NH₂, —CH═NR¹¹, or —C(═O)—R¹¹ where:

R⁸ is O or S;

R⁹ represents N₃, SH₂, NH₂, NO₂, or OLR¹², and

R¹⁰ represents lower alkyl, amino, OLR¹², or a pharmaceutically acceptable salt thereof, or

R⁹ and R¹⁰, together with the phosphorus to which they are attached, form a 5- to 8-membered heterocyclic ring;

R¹¹ represents H, alkyl, alkenyl, alkynyl, —NH₂, —(CH₂)ₚ—R¹², —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², (CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)NH₂, —C(O)OR¹³, or —C(Z¹)(Z²)(Z³);

R¹² represents H, alkyl, alkenyl, aryl, heteroaryl, cycloalkyl, cycloalkenyl, or heterocyclyl;

R¹³ represents H, alkyl, alkenyl, or LR¹²;

Z¹ represents a halogen;

Z² and Z³ independently represent H or halogen;

p is, independently for each occurrence, an integer from 0 to 8; and

q is, independently for each occurrence, an integer from 1 to 8.

In certain preferred embodiments, R⁶ represents CN, CHO, or C(═O)C(Z¹)(Z²)(Z³), where Z¹ represents a halogen, and Z² and Z³ represent H or halogen. In another embodiment, R⁶ represents C(═O)C(Z¹)(Z²)(Z³), where Z¹ represents fluorine, and Z² and Z³ represent H or fluorine.

In certain preferred embodiments, R⁶ represents a group of formula —B(Y¹)(Y²), wherein Y¹ and Y² are independently OH or a group that is hydrolysable to OH, or together with the boron atom to which they are attached form a 5- to 8-membered ring that is hydrolysable to a boronic acid.

Another aspect of the invention relates to a protease inhibitor having a structure of Formula III

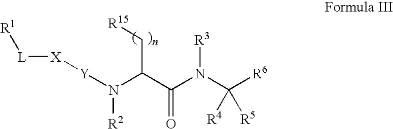

or pharmaceutically acceptable salt thereof, where:

R¹ represents H, alkyl, alkoxy, alkenyl, alkynyl, amino, alkylamino, acylamino, cyano, sulfonylamino, acyloxy, aryl, cycloalkyl, heterocyclyl, heteroaryl, or a polypeptide chain of 1 to 8 amino acid residues;

R² represents H, lower alkyl, or aralkyl;

R³ and R⁴ independently represent H, halogen, or alkyl, or R³ and R⁴ together with the carbon to which they are attached, form a 3- to 6-membered heterocyclic ring;

R⁵ represents H, halogen, lower alkyl, or aralkyl, preferably H or lower alkyl;

R⁶ represents a functional group that reacts with an active site residue of a targeted protease to form a covalent adduct;

R⁷ represents H, aryl, alkyl, aralkyl, cycloalkyl, heterocyclyl, heteroaryl, heteroaralkyl, or a polypeptide chain of 1 to 8 amino acid residues;

R¹⁵ is a functional group that has either a positive or negative charge at physiological pH, preferably an amine or carboxylic acid;

L is absent or represents alkyl, alkenyl, alkynyl, —(CH₂)ₘO(CH₂)ₘ—, —(CH₂)ₘNR₂(CH₂)ₘ—, and —(CH₂)ₘS(CH₂)ₘ—;

X is absent or represents —N(R⁷)—, —O—, or —S—;

Y is absent or represents —C(═O)—, —C(═S)—, or —SO₂—;

m is, independently for each occurrence, an integer from 0 to 10; and

n is an integer from 1 to 6.

In certain preferred embodiments, R¹ represents H or lower alkyl, R³ is H and R⁴ is lower alkyl, or R³ and R⁴ together with the carbon to which they are attached form a 5-membered ring, and n is an integer from 1 to 4.

In certain other preferred embodiments R¹ represents H or lower alkyl, R³ represents H, R⁴ represents H or lower alkyl, R⁵ represents H, and n is an integer from 1 to 4.

In certain preferred embodiments where X, Y, and L are absent, R¹ is a polypeptide chain of 2 to 8 amino acid residues, where proline is the residue that is directly attached to the leftmost residue of Formula II. In certain such embodiments, R¹ is a polypeptide chain of 2 amino acid residues, wherein proline is the residue that is directly attached to the leftmost nitrogen of Formula II.

In certain preferred embodiments, n is an integer from 1 to 4 and R¹⁵ is a functional group that has either a positive or negative charge at physiological pH. In more preferred embodiments, n is an integer from 1 to 4 and R¹⁵ is selected from amine, carboxylic acid, imidazole, and guanidine functionality.

In certain embodiments, R⁶ represents boronic acid, —CN, —SO₂Z¹, —P(═O)Z¹, —P(═R⁸)R⁹R¹⁰, —C(═NH)NH₂, —CH═NR¹¹, or —C(═O)—R¹¹ where:

R⁸ is O or S;

R⁹ represents N₃, SH₂, NH₂, NO₂, or OLR¹², and

R¹⁰ represents lower alkyl, amino, OLR¹², or a pharmaceutically acceptable salt thereof, or

R⁹ and R¹⁰, together with the phosphorus to which they are attached, form a 5- to 8-membered heterocyclic ring;

R¹¹ represents H, alkyl, alkenyl, alkynyl, NH₂, —(CH₂)ₚ—R¹², (CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)NH₂, —C(O)OR¹³, or —C(Z¹)(Z²)(Z³);

R¹² represents H, alkyl, alkenyl, aryl, heteroaryl, cycloalkyl, cycloalkenyl, or heterocyclyl;

R¹³ represents H, alkyl, alkenyl, or LR¹²;

Z¹ represents a halogen;

Z² and Z³ independently represent H or halogen;

p is, independently for each occurrence, an integer from 0 to 8; and

q is, independently for each occurrence, an integer from 1 to 8.

In certain preferred embodiments, R⁶ represents CN, CHO, or C(═O)C(Z¹)(Z²)(Z³), wherein Z¹ represents a halogen, and Z² and Z³ represent H or halogen. In another embodiment, R⁶ represents C(═O)C(Z¹)(Z²)(Z³), wherein Z¹ represents fluorine, and Z² and Z³ represent H or fluorine.

In certain preferred embodiments, R⁶ represents a group of formula —B(Y¹)(Y²), wherein Y¹ and Y² are independently OH or a group that is hydrolysable to OH, or together with the boron atom to which they are attached form a 5- to 8-membered ring that is hydrolysable to a boronic acid.

Yet another aspect of the invention relates to a protease inhibitor having a structure of Formula IV:

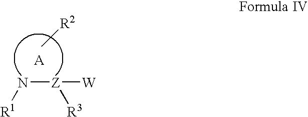

or a pharmaceutically acceptable salt thereof, where

A is selected from a 4-8 membered heterocycle including the N and a Cα carbon;

Z is C or N;

W is selected from CN, —CH═NR⁵, a functional group which reacts with an active site residue of the targeted protease,

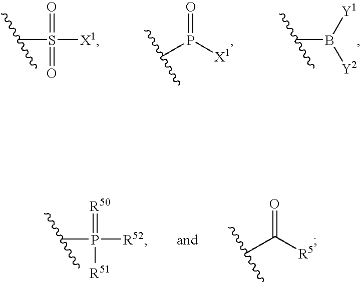

R¹ is selected from a C-terminally linked amino acid residue or amino acid analog, a C-terminally linked peptide or peptide analog, an amino-protecting group,

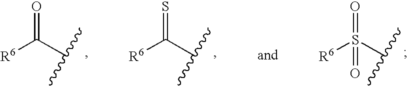

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl, thiocarbonyl, amino, acylamino, amido, cyano, nitro, azido, sulfate, sulfonate, sulfonamido, —(CH₂)ₘ—R⁷, (CH₂)ₘ—OH, —(CH₂)ₘ—O-lower alkyl, —(CH₂)ₘ—O-lower alkenyl, —(CH₂)ₘ—O—(CH₂)ₘ—R⁷, —(CH₂)ₘ—SH, —(CH₂)ₘ—S-lower alkyl, —(CH₂)ₘ—S-lower alkenyl, or —(CH₂)ₙ—S—(CH₂)ₘ—R⁷, wherein at least one R² is selected from —OH, lower alkyl, lower alkoxy, lower hydroxyalkyl, and lower alkoxyalkyl, preferably at least one of lower alkyl (e.g., methyl), lower alkoxy, lower hydroxyalkyl (e.g., hydroxymethyl), and lower alkoxyalkyl;

when Z is N, R³ is hydrogen;

when Z is C, R³ is selected from hydrogen, halogen, lower alkyl, lower alkenyl, lower alkynyl, carbonyl, thiocarbonyl, amino, acylamino, amido, cyano, nitro, azido, sulfate, sulfonate, sulfonamido, —(CH₂)ₘ—R⁷, —(CH₂)ₘ—OH, —(CH₂)ₘ—O-lower alkyl, —(CH₂)ₘ—O-lower alkenyl, —(CH₂)ₙ—O—(CH₂)ₘ—R⁷, (CH₂)ₘ—SH, —(CH₂)ₘ—S-lower alkyl —(CH₂)ₘ—S-lower alkenyl, and —(CH₂)ₙ—S—(CH₂)ₘ—R⁷;

R⁵ is selected from hydrogen, alkyl, alkenyl, alkynyl, —C(X¹)(X²)X³, —(CH₂)ₘ—R⁷, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R⁷, —(CH₂)ₙ—SH, —(CH₂)ₙ—S-alkyl, —(CH₂)ₙ—S-alkenyl, —(CH₂)—S-alkynyl, —(CH₂)—S—(CH₂)ₘ—R⁷, —C(O)C(O)NH₂, and —C(O)C(O)OR⁷;

R⁶ is selected from hydrogen, halogen, alkyl, alkenyl, alkynyl, aryl, —(CH₂)ₘ—R⁷, —(CH₂)ₘ—OH, —(CH₂)ₘ—O-alkyl, —(CH₂)ₘ—O-alkenyl, —(CH₂)ₘ—O-alkynyl, —(CH₂)ₘ—O—(CH₂)ₘ—R⁷, —(CH₂)ₘ—SH, —(CH₂)ₘ—S-alkyl, —(CH₂)ₘ—S-alkenyl, —(CH₂)ₘ—S-alkynyl, or —(CH₂)ₘ—S—(CH₂)ₘ—R⁷,

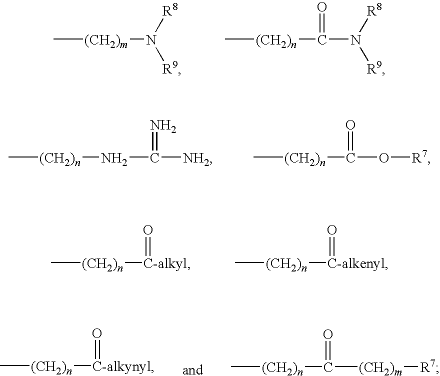

each R⁷ is independently selected from aryl, aralkyl, cycloalkyl, cycloalkenyl, and heterocyclyl;

each R^{7′} is independently selected from hydrogen, alkyl, alkenyl, aryl, aralkyl, cycloalkyl, cycloalkenyl and heterocyclyl;

R⁸ and R⁹ are each independently selected from hydrogen, alkyl, alkenyl, —(CH₂)ₘ—R⁷, —C(═O)-alkyl, —C(═O)-alkenyl, —C(═O)-alkynyl, and —C(═O)—(CH₂)ₘ—R⁷; or

R⁸ and R⁹ taken together with the N atom to which they are attached complete a heterocyclic ring having from 4 to 8 atoms in the ring structure;

R⁵⁰ is O or S;

R⁵¹ is selected from N₃, SH, NH₂, NO₂, and OR^{7′};

R⁵² is selected from hydrogen, lower alkyl, amine, OR^{7′}, or a pharmaceutically acceptable salt thereof, or

R⁵¹ and R⁵² taken together with the P atom to which they are attached complete a heterocyclic ring having from 5 to 8 atoms in the ring structure;

X¹ is a halogen;

X² and X³ are each selected from hydrogen and halogen;

Y¹ and Y² are each independently selected from OH and a group capable of being hydrolyzed to OH, including cyclic derivatives where Y¹ and Y² are connected via a ring having from 5 to 8 atoms in the ring structure;

m is zero or an integer in the range of 1 to 8; and

n is an integer in the range of 1 to 8.

In certain above embodiments, the protease inhibitor inhibits DPIV with a Kᵢ of 50 nm or less.

In certain embodiments, the inhibitor is orally active.

In certain embodiments, the inhibitor has a therapeutic index in humans of at least 2, and even more preferably 5, 10 or even 100, e.g., such as a therapeutic index for regulating glucose metabolism.

Another aspect of the invention provides a pharmaceutical composition comprising a pharmaceutically acceptable carrier and one or more of the subject protease inhibitors, or a pharmaceutically acceptable salt or prodrug thereof.

Another aspect of the invention provides for use of one or more of the subject inhibitors in the manufacture of a medicament for inhibiting a post-proline cleaving enzyme in vivo. For example, the subject inhibitors can be used to manufacture medicaments for increasing plasma concentrations of one or more peptide hormones processed by post-proline cleaving enzymes (e.g., DP-IV and the like). Exemplary medicaments are useful in increasing plasma concentrations of such hormones as glucagons-like peptide, NPY, PPY, secretin, GLP-1, GLP-2, and GIP.

In certain preferred embodiments, the subject inhibitors can be used to manufacture medicaments for regulating glucose metabolism, such as for use in treating patients suffering from Type II diabetes, insulin resistance, glucose intolerance, hyperglycemia, hypoglycemia, hyperinsulinemia, obesity, hyperlipidemia, or hyperlipoproteinemia.

Yet another aspect of the invention provides a packaged pharmaceutical comprising: a preparation of one or more of the subject protease inhibitors; optionally a pharmaceutically acceptable carrier; and instructions, written and/or pictorial, describing the use of the preparation for inhibiting a post-proline cleaving enzyme in vivo, such as for regulating glucose metabolism.

The packaged pharmaceutical can also include, e.g., as a co-formulation with the protease inhibitor or simply co-packaged with the protease inhibitor, insulin and/or an insulinotropic agent.

The packaged pharmaceutical can also include, e.g., as a co-formulation with the protease inhibitor or simply co-packaged with the protease inhibitor, an Ml receptor antagonist, a prolactin inhibitor, agents acting on the ATP-dependent potassium channel of β-cells, metformin, and/or glucosidase inhibitors.

The present invention also relates to improved methods for the long-term reduction and abatement of at least one of the foregoing disorders based on a therapeutic regimen administered over the short-term.

The present invention further provides a method for regulating and altering on a long-term basis the glucose and lipogenic responses of vertebrate animals, including humans.

In particular, the compounds of the invention may be employed to provide methods for producing long-lasting beneficial changes in one or more of the following: the sensitivity of the cellular response of a species to insulin (reduction of insulin resistance), blood insulin levels, hyperinsulinemia, blood glucose levels, the amount of body fat stores, and blood lipoprotein levels, thus providing effective treatments for diabetes, obesity and/or atherosclerosis.

### BRIEF DESCRIPTION OF THE FIGURES

FIG. 1 shows the inhibition of DPIV by Lys-boroPro over 120 minutes at three different doses.

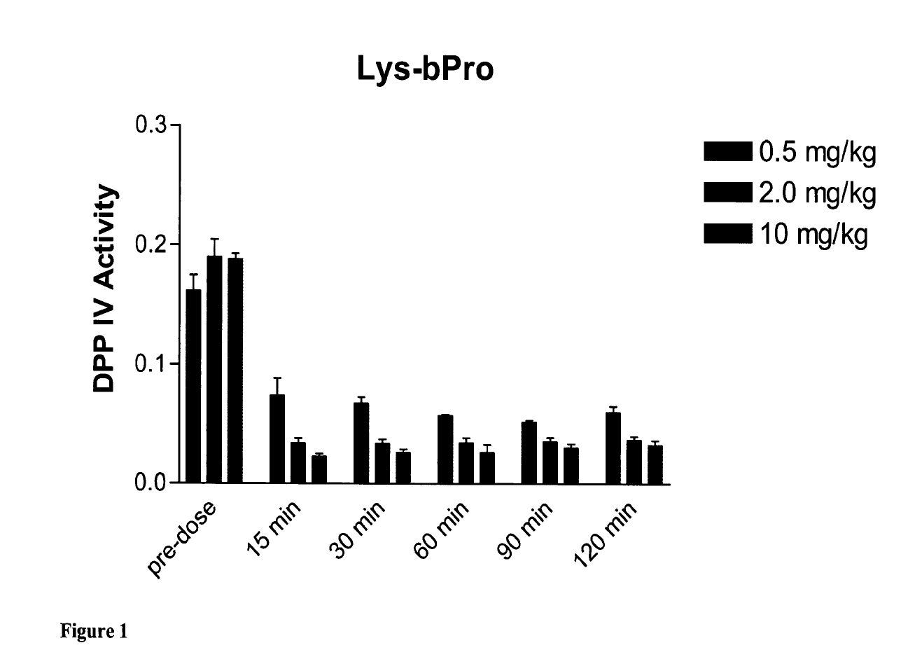

FIG. 2 shows the inhibition of DPIV by Arg-boroPro over 120 minutes at three different doses.

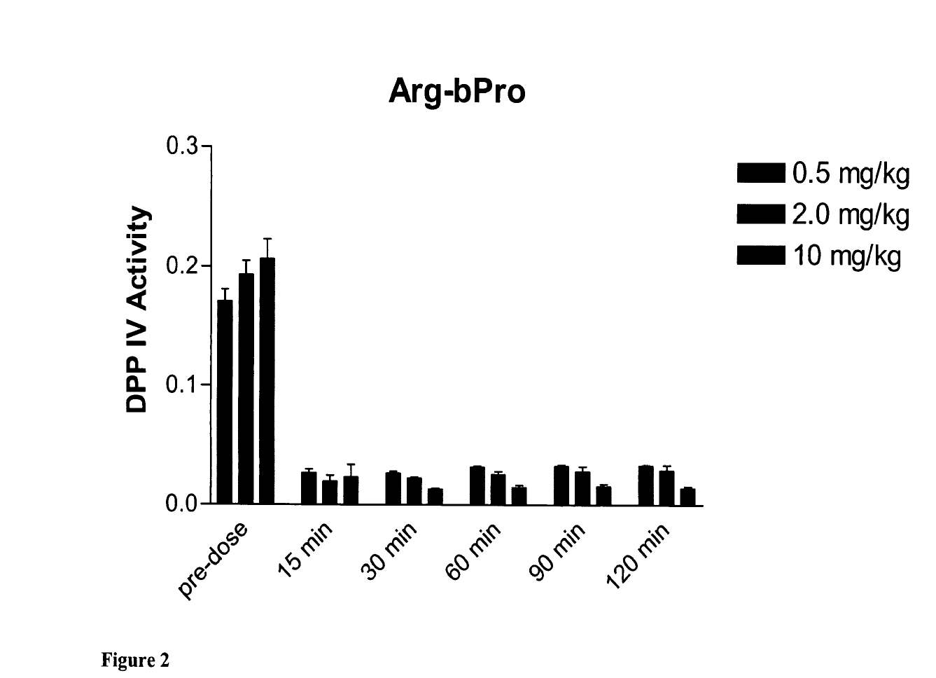

### DETAILED DESCRIPTION

### I. Overview

The present invention relates to inhibitors of post-proline cleaving enzymes (PPCE), such as inhibitors of dipeptidyl peptidase IV, as well as pharmaceutical compositions thereof, and methods for using such inhibitors. The prototype of these molecules has an acidic amino acid and an electrophilic site carrying a variety of side chains.

Salient features for compounds of the present invention include: better therapeutic indices, owing in part to reduced toxicity and/or improved specificity for the targeted protease; better oral availability; increased shelf-life; and/or increased duration of action (such as single oral dosage formulations which are effective for more than 4 hours, and even more preferably for more than 8, 12, or 16 hours).

The compounds of the present invention can be used as part of treatments for a variety of disorders/conditions, such as those which are mediated by DPIV. For instance, the subject inhibitors can be used to up-regulate GIP and GLP-1 activities, e.g., by increasing the half-life of those hormones, as part of a treatment for regulating glucose levels and/or metabolism, e.g., to reduce insulin resistance, treat hyperglycemia, hyperinsulinemia, obesity, hyperlipidemia, hyperlipoproteinemia (such as chylomicrons, VLDL and LDL), and to regulate body fat and more generally lipid stores, and, more generally, for the improvement of metabolism disorders, especially those associated with diabetes, obesity and/or atherosclerosis.

While not wishing to be bound by any particular theory, it is observed that compounds which inhibit DPIV are, correlatively, able to improve glucose tolerance, though not necessarily through mechanisms involving DPIV inhibition per se. Indeed, similar compounds have been shown to be effective in mice lacking a GLP-1 receptor suggesting that the subject method may not include a mechanism of action directly implicating GLP-1 itself, though it has not been ruled out that GLP-1 may have other receptors. However, in light of the correlation with DPIV inhibition, in preferred embodiments, the subject method utilizes an agent with a Kᵢ for DPIV inhibition of 50.0 nm or less, more preferably of 10.0 nm or less, and even more preferably of 1.0, 0.1, or even 0.01 nM or less. Indeed, inhibitors with Kᵢ values in the picomolar and even femtomolar range are contemplated. Thus, while the active agents are described herein, for convenience, as “DPIV inhibitors”, it will be understood that such nomenclature is not intending to limit the subject invention to a particular mechanism of action.

Certain of the subject compounds have extended duration. Accordingly, in certain preferred embodiments, the inhibitor(s) is selected, and the amount of inhibitor formulated, to provide a dosage which inhibits serum PPCE (e.g., DPIV) levels by at least 50 percent for at least 4 hours after a single dose, and even more preferably for at least 8 hours or even 12 or 16 hours after a single dose.

For instance, in certain embodiments the method involves administration of a DPIV inhibitor, preferably at a predetermined time(s) during a 24-hour period, in an amount effective to improve one or more aberrant indices associated with glucose metabolism disorders (e.g., glucose intolerance, insulin resistance, hyperglycemia, hyperinsulinemia, and Type I and II diabetes).

In other embodiments, the method involves administration of a DPIV inhibitor in an amount effective to improve aberrant indices associated with obesity. Fat cells release the hormone leptin, which travels in the bloodstream to the brain and, through leptin receptors there, stimulates production of GLP-1. GLP-1, in turn, produces the sensation of being full. The leading theory is that the fat cells of most obese people probably produce enough leptin, but leptin may not be able to properly engage the leptin receptors in the brain, and so does not stimulate production of GLP-1. There is accordingly a great deal of research towards utilizing preparations of GLP-1 as an appetite suppressant. The subject method provides a means for increasing the half-life of both endogenous and ectopically added GLP-1 in the treatment of disorders associated with obesity.

In a more general sense, the present invention provides methods and compositions for altering the pharmacokinetics of a variety of different polypeptide hormones by inhibiting the proteolysis of one or more peptide hormones by DPIV or some other proteolytic activity. Post-secretory metabolism is an important element in the overall homeostasis of regulatory peptides, and the other enzymes involved in these processes may be suitable targets for pharmacological intervention by the subject method.

For example, the subject method can be used to increase the half-life of other proglucagon-derived peptides, such as glicentin (corresponding to PG 1-69), oxyntomodulin (PG 33-69), glicentin-related pancreatic polypeptide (GRPP, PG 1-30), intervening peptide-2 (IP-2, PG 111-122 amide), and glucagon-like peptide-2 (GLP-2, PG 126-158).

GLP-2, for example, has been identified as a factor responsible for inducing proliferation of intestinal epithelium. See, for example, Drucker et al. (1996) PNAS 93:7911. The subject method can be used as part of a regimen for treating injury, inflammation or resection of intestinal tissue, e.g., where enhanced growth and repair of the intestinal mucosal epithelial is desired, such as in the treatment of Crohn's disease or Inflammatory Bowel Disease (IBD).

DPIV has also been implicated in the metabolism and inactivation of growth hormone-releasing factor (GHRF). GHRF is a member of the family of homologous peptides that includes glucagon, secretin, vasoactive intestinal peptide (VIP), peptide histidine isoleucine (PHI), pituitary adenylate cyclase activating peptide (PACAP), gastric inhibitory peptide (GIP), and helodermin (Kubiak et al. (1994) Peptide Res 7:153). GHRF is secreted by the hypothalamus, and stimulates the release of growth hormone (GH) from the anterior pituitary. Thus, the subject method can be used to improve clinical therapy for certain growth hormone deficient children, and in clinical therapy of adults to improve nutrition and to alter body composition (muscle vs. fat). The subject method can also be used in veterinary practice, for example, to develop higher yield milk production and higher yield, leaner livestock.

Likewise, the DPIV inhibitors of the subject invention can be used to alter the plasma half-life of secretin, VIP, PHI, PACAP, GIP, and/or helodermin. Additionally, the subject method can be used to alter the pharmacokinetics of Peptide YY and neuropeptide Y, both members of the pancreatic polypeptide family, as DPIV has been implicated in the processing of those peptides in a manner which alters receptor selectivity.

In other embodiments, the subject inhibitors can be used to stimulate hematopoiesis.

In still other embodiments, the subject inhibitors can be used to inhibit growth or vascularization of transformed cells/tissues, e.g., to inhibit cell proliferation such as that associated with tumor growth and metastasis, and for inhibiting angiogenesis in an abnormal proliferative cell mass.

In yet other embodiments, the subject inhibitors can be used to reduce immunological responses, e.g., as an immunosuppressant.

In yet other examples, the DPIV inhibitors according to the present invention can be used to treat CNS maladies such as strokes, tumors, ischemia, Parkinson's disease, memory loss, hearing loss, vision loss, migraines, brain injury, spinal cord injury, Alzheimer's disease, and amyotrophic lateral sclerosis (which has a CNS component). Additionally, the DPIV inhibitors can be used to treat disorders having a more peripheral nature, including multiple sclerosis and diabetic neuropathy.

Another aspect of the present invention relates to pharmaceutical compositions of the subject post-proline cleaving enzyme inhibitors, particularly DPIV inhibitors, and their uses in treating and/or preventing disorders which can be improved by altering the homeostasis of peptide hormones. In a preferred embodiment, the inhibitors have hypoglycemic and antidiabetic activities, and can be used in the treatment of disorders marked by aberrant glucose metabolism (including storage). In particular embodiments, the compositions of the subject methods are useful as insulinotropic agents, or to potentiate the insulinotropic effects of such molecules as GLP-1. In this regard, certain embodiments of the present compositions can be useful for the treatment and/or prophylaxis of a variety of disorders, including one or more of: hyperlipidemia, hyperglycemia, obesity, glucose tolerance insufficiency, insulin resistance, and diabetic complications.

In general, the inhibitors of the subject method are small molecules, e.g., with molecular weights less than 7500 amu, preferably less than 5000 amu, and even more preferably less than 2000 or even less than 1000 amu. In preferred embodiments, the inhibitors are orally active.

### II. Definitions

The term “high affinity” as used herein means strong binding affinity between molecules with a dissociation constant K_{D} of no greater than 1 μM. In a preferred case, the K_{D} is less than 100 nM, 10 nM, 1 nM, 100 pM, or even 10 pM or less. In a most preferred embodiment, the two molecules can be covalently linked (K_{D} is essentially 0).

The term “boro-Ala” refers to the analog of alanine in which the carboxylate group (COOH) is replaced with a boronyl group (B(OH)₂). Likewise, the term “boro-Pro” refers to the analog of proline in which the carboxylate group (COOH) is replaced with a boronyl group (B(OH)₂). More generally, the term “boro-Xaa”, where Xaa is an amino acid residue, refers to the analog of an amino acid in which the carboxylate group (COOH) is replaced with a boronyl group (B(OH)₂).

A “patient” or “subject” to be treated by the subject method can mean either a human or non-human subject.

The term “ED₅₀” means the dose of a drug that, in 50% of patients, will provide a clinically relevant improvement or change in a physiological measurement, such as glucose responsiveness, increase in hematocrit, decrease in tumor volume, etc.

The term “IC₅₀” means the dose of a drug that inhibits a biological activity by 50%, e.g., the amount of inhibitor required to inhibit at least 50% of DPIV (or other PPCE) activity in vivo.

A compound is said to have an “insulinotropic activity” if it is able to stimulate, or cause the stimulation of, the synthesis or expression of the hormone insulin.

The term “interact” as used herein is meant to include all interactions (e.g., biochemical, chemical, or biophysical interactions) between molecules, such as protein-protein, protein-nucleic acid, nucleic acid-nucleic acid, protein-small molecule, nucleic acid-small molecule, or small molecule-small molecule interactions.

The term “LD₅₀” means the dose of a drug that is lethal in 50% of test subjects.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “therapeutic index” refers to the therapeutic index of a drug defined as LD₅₀/ED₅₀.

A “therapeutically effective amount” of a compound, e.g., such as a DPIV inhibitor of the present invention, with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

A “single oral dosage formulation” is a dosage which provides an amount of drug to produce a serum concentration at least as great as the EC₅₀ for that drug, but less than the LD₅₀. Another measure for a single oral dosage formulation is that it provides an amount of drug necessary to produce a serum concentration at least as great as the IC₅₀ for that drug, but less than the LD₅₀. By either measure, a single oral dosage formulation is preferably an amount of drug which produces a serum concentration at least 10 percent less than the LD₅₀, and even more preferably at least 50 percent, 75 percent, or even 90 percent less than the drug's the LD₅₀.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

Alkyl refers to a fully saturated branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6, or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, a cyano, a nitro, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl, and sulfonate), and silyl groups, as well as ethers, alkylthios, carbonyls (including ketones, aldehydes, carboxylates, and esters), —CF₃, —CN, and the like. Exemplary substituted alkyls are described below. Cycloalkyls can be further substituted with alkyls, alkenyls, alkoxyls, alkylthios, aminoalkyls, carbonyl-substituted alkyls, —CF₃, —CN, and the like.

Unless the number of carbons is otherwise specified, “lower alkyl”, as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur moiety attached thereto. In preferred embodiments, the “alkylthio” moiety is represented by one of —(S)-alkyl, —(S)-alkenyl, —(S)-alkynyl, and —(S)—(CH₂)ₘ, —R¹, wherein m and R¹ are defined below. Representative alkylthio groups include methylthio, ethylthio, and the like.

Alkenyl refers to any branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

Alkynyl refers to hydrocarbyl moieties of the scope of alkenyl, but having one or more triple bonds in the moiety.

The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R¹, where m and R₁ are described below.

The terms “amine” and “amino” are art-recognized and refer to both unsubstituted and substituted amines, e.g., a moiety that can be represented by the general formulae:

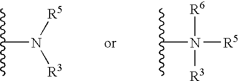

wherein R³, R⁵ and R⁶ each independently represent a hydrogen, an alkyl, an alkenyl, —(CH₂)ₘ—R¹, or R³ and R⁵ taken together with the N atom to which they are attached complete a heterocycle having from 4 to 8 atoms in the ring structure; R¹ represents an alkenyl, aryl, cycloalkyl, a cycloalkenyl, a heterocyclyl, or a polycyclyl; and m is zero or an integer in the range of 1 to 8. In preferred embodiments, only one of R³ or R⁵ can be a carbonyl, e.g., R³, R⁵, and the nitrogen together do not form an imide. In even more preferred embodiments, R³ and R⁵ (and optionally R⁶) each independently represent a hydrogen, an alkyl, an alkenyl, or —(CH₂)ₘ—R¹. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a conjugate acid with a pKₐ≧7.00, i.e., the protonated forms of these functional groups have pKₐs relative to water above about 7.00.

The term “carbonyl” is art-recognized and includes such moieties as can be represented by the general formula:

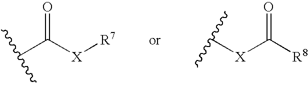

wherein X is a bond or represents an oxygen or a sulfur, and R⁷ represents a hydrogen, an alkyl, an alkenyl, —(CH₂)ₘ—R¹ or a pharmaceutically acceptable salt, R⁸ represents a hydrogen, an alkyl, an alkenyl or —(CH₂)ₘ—R¹, where m and R¹ are as defined above. Where X is an oxygen and R⁷ or R⁸ is not hydrogen, the formula represents an “ester”. Where X is an oxygen, and R⁷ is as defined above, the moiety is referred to herein as a carboxyl group, and particularly when R⁷ is a hydrogen, the formula represents a “carboxylic acid”. Where X is an oxygen, and R⁸ is a hydrogen, the formula represents a “formate”. In general, where the oxygen atom of the above formula is replaced by a sulfur, the formula represents a “thiocarbonyl” group. Where X is a sulfur and R⁷ or R⁸ is not hydrogen, the formula represents a “thioester” group. Where X is a sulfur and R⁷ is a hydrogen, the formula represents a “thiocarboxylic acid” group. Where X is a sulfur and R⁸ is a hydrogen, the formula represents a “thioformate” group. On the other hand, where X is a bond, and R⁷ is not hydrogen, the above formula represents a “ketone” group. Where X is a bond, and R⁷ is a hydrogen, the above formula represents an “aldehyde” group.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “hydrocarbyl” refers to a monovalent hydrocarbon moiety comprised of carbon chains or rings of up to 26 carbon atoms to which hydrogen atoms are attached. The term includes alkyl, cycloalkyl, alkenyl, alkynyl, and aryl groups, groups which have a mixture of saturated and unsaturated bonds, carbocyclic rings, and includes combinations of such groups. It may refer to straight chain, branched-chain, cyclic structures, or combinations thereof.

The term “hydrocarbylene” refers to a divalent hydrocarbyl moiety. Representative examples include alkylene, phenylene, or cyclohexylene. Preferably, the hydrocarbylene chain is fully saturated and/or has a chain of 1 to 10 carbon atoms.

As used herein, the term “nitro” means —NO₂; the term “halogen” designates —F, —Cl, —Br, or —I; the term “sulfhydryl” means —SH; the term “hydroxyl” means —OH; and the term “sulfonyl” means —SO₂—.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

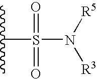

in which R³ and R⁵ are as defined above.

The term “sulfate” is art recognized and includes a moiety that can be represented by the general formula:

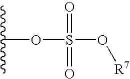

in which R⁷ is as defined above.

The term “sulfonamide” is art recognized and includes a moiety that can be represented by the general formula:

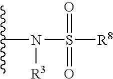

in which R³ and R⁸ are as defined above.

The term “sulfonate” is art-recognized and includes a moiety that can be represented by the general formula:

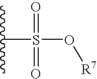

in which R⁷ is an electron pair, hydrogen, alkyl, cycloalkyl, or aryl.

The terms “sulfoxido” or “sulfinyl”, as used herein, refers to a moiety that can be represented by the general formula:

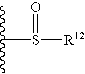

in which R¹² is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls, or alkynyls.

As used herein, the definition of each expression, e.g., alkyl, m, n, etc., when it occurs more than once in any structure, is intended to be independent of its definition elsewhere in the same structure.

A “small” substituent is one of 10 atoms or less.

The terms “amino acid residue” and “peptide residue” mean an amino acid or peptide molecule without the —OH of its carboxyl group. In general the abbreviations used herein for designating the amino acids and the protective groups are based on recommendations of the IUPAC-IUB Commission on Biochemical Nomenclature (see Biochemistry (1972) 11:1726-1732). For instance Met, Ile, Leu, Ala, and Gly represent “residues” of methionine, isoleucine, leucine, alanine, and glycine, respectively. Residue means a moiety derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H-(the side chain of glycine).

For the most part, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs which have been identified as constituents of peptidylglycan bacterial cell walls.

The term amino acid residue further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated, the amino acid or residue can have the configuration (D), (L), or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) and (L) stereoisomers.

The phrase “protecting group” as used herein means substituents which protect the reactive functional group from undesirable chemical reactions. Examples of such protecting groups include esters of carboxylic acids and boronic acids, ethers of alcohols, and acetals and ketals of aldehydes and ketones. For instance, the phrase “N-terminal protecting group” or “amino-protecting group” as used herein refers to various amino-protecting groups which can be employed to protect the N-terminus of an amino acid or peptide against undesirable reactions during synthetic procedures. Examples of suitable groups include acyl protecting groups such as, to illustrate, formyl, dansyl, acetyl, benzoyl, trifluoroacetyl, succinyl, and methoxysuccinyl; aromatic urethane protecting groups as, for example, benzyloxycarbonyl (Cbz); and aliphatic urethane protecting groups such as t-butoxycarbonyl (Boc) or 9-Fluorenylmethoxycarbonyl (Fmoc).

As noted above, certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments where a particular enantiomer is preferred, a compound of the present invention is enriched to have >60%, >70%, >80%, >90%, >95%, or even greater than 98% or 99% of the preferred enantiomer, as opposed to a racemate where the two enantiomers each are present to the extent of 50%.

If, for instance, a particular enantiomer of a compound of the present invention is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

A compound is said to have an “insulinotropic activity” if it is able to stimulate, or cause the stimulation of, the synthesis or expression of the hormone insulin.

It will be understood that all generic structures recited herein, with respect to appropriate combinations of substituents, are intended to cover those embodiments permitted by valency and stability.

### III. Exemplary Embodiments

(i). Compounds

Useful compounds will be described below using various formulae. In each case, the variables in the formula are defined specifically for each individual formulae. A definition of a variable for one formula should not be used to vary a definition provided for another formula, although a variable that has not been defined for one formula may be interpreted by analogy with a definition elsewhere for a similar formula.

In certain embodiments of the invention, a subject compound has a structure of Formula I

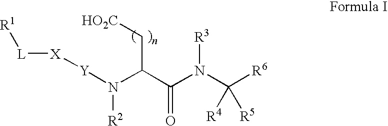

wherein

R¹ represents H, alkyl, alkoxy, alkenyl, alkynyl, amino, alkylamino, acylamino, cyano, sulfonylamino, acyloxy, aryl, cycloalkyl, heterocyclyl, heteroaryl, or a polypeptide chain of 1 to 8 amino acid residues;

R² represents H, lower alkyl, or aralkyl;

R³ and R⁴ independently represent H, halogen, or alkyl, or R³ and R⁴ together with the atoms to which they are attached, form a 3- to 6-membered heterocyclic ring;

R⁵ represents H, halogen, lower alkyl, or aralkyl;

R⁶ represents a functional group that reacts with an active site residue of a targeted protease to form a covalent adduct;

R⁷ represents H, aryl, alkyl, aralkyl, cycloalkyl, heterocyclyl, heteroaryl, heteroaralkyl, or polypeptide chains of 1 to 8 amino acid residues;

L is absent or represents alkyl, alkenyl, alkynyl, —(CH₂)ₘO(CH₂)ₘ—, —(CH₂)ₘNR₂(CH₂)ₘ—, and —(CH₂)ₘS(CH₂)ₘ—;

X is absent or represents —N(R⁷)—, —O—, or —S—;

Y is absent or represents —C(═O)—, —C(═S)—, or —SO₂—;

m is, independently for each occurrence, an integer from 0 to 10; and

n is an integer from 1 to 6.

In certain preferred embodiments, R¹ represents H or lower alkyl, R³ and R⁴ together with the atoms to which they are attached form a 5-membered ring, and n is 2.

In certain other preferred embodiments R¹ represents H or lower alkyl, R³ represents H, R⁴ represents H or lower alkyl, R⁵ represents H, and n is 2.

In certain preferred embodiments, R¹ is a polypeptide chain of 2 to 8 amino acid residues, wherein proline is the residue that is directly attached. Most preferably R¹ is a polypeptide chain of 2 amino acid residues

In certain above embodiments, R⁶ represents cyano, boronic acid, —SO₂Z¹, —P(═O)Z¹, —P(═R⁸)R⁹R¹⁰, —C(═NH)NH₂, —CH═NR¹¹, and —C(═O)—R¹¹, wherein

R⁸ represents O or S;

R⁹ represents N₃, SH₂, NH₂, NO₂, and OLR¹², and

R¹⁰ represents lower alkyl, amino, OLR¹², or a pharmaceutically acceptable salt thereof, or

R⁹ and R¹⁰, together with the phosphorus to which they are attached, form a 5- to 8-membered heterocyclic ring;

R¹¹ represents H, alkyl, alkenyl, alkynyl, —(CH₂)ₚ—R¹², —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)C(O)NH₂, —C(O)C(O)OR¹³, or —C(Z¹)(Z²)(Z³);

R¹² represents H, alkyl, alkenyl, aryl, cycloalkyl, cycloalkenyl, and heterocyclyl;

R¹³ represents H, alkyl, alkenyl, and LR¹²;

Z¹ represents a halogen;

Z² and Z³ independently represent H or halogen;

p is, independently for each occurrence, an integer from 0 to 8; and

q is, independently for each occurrence, an integer from 1 to 8.

In another embodiment, R⁶ represents CN, CHO, or C(═O)C(Z¹)(Z²)(Z³), wherein Z¹ represents a halogen, and Z² and Z³ represent H or halogen. In certain such embodiments, R⁶ represents C(═O)C(Z¹)(Z²)(Z³), wherein Z¹ represents fluorine, and Z² and Z³ represent H or fluorine.

In certain preferred embodiments, R⁶ represents a group of formula —B(Y¹)(Y²), wherein Y¹ and Y² are independently OH or a group that is hydrolysable to OH (i.e., thereby forming a boronic acid), or together with the boron atom to which they are attached form a 5- to 8-membered ring that is hydrolysable to a boronic acid.

In certain preferred embodiments, R³ and R⁴ together with the atoms to which they are attached form a 5-membered ring, which is substituted with one or more groups selected from hydroxyl, lower alkyl (e.g., methyl), lower alkenyl, lower alkynyl, lower alkoxy, lower hydroxyalkyl (e.g., hydroxymethyl), and lower alkoxyalkyl.

In more preferred embodiments, the substituent group is selected from lower alkyl, lower hydroxyalkyl and lower alkoxyalkyl. In more preferred such embodiments, the substituent group is located at the 5-position of the ring.

In other more preferred embodiments, the substituent group is hydroxyl, which is preferably located at the 4-position of the ring.

In certain embodiments, the substituent group on the 5-membered ring containing R³ and R⁴ is selected from lower alkyl (e.g., methyl), hydroxyl, lower hydroxyalkyl (e.g., hydroxymethyl) and lower alkoxyalkyl. In certain preferred such embodiments, the substituent group has a cis-stereochemical relationship to R⁶. Such stereochemical relationships are particularly advantageous for compounds having substituents at the 4- or 5-position of the 5-membered ring, as discussed immediately above.

Exemplary structures include

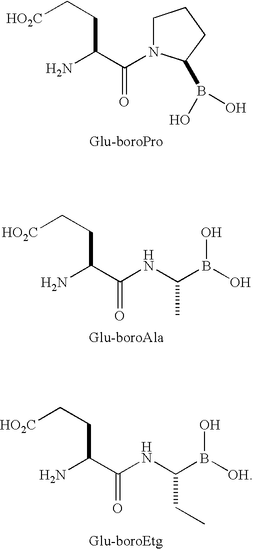

In certain embodiments of the invention, a subject compound has a structure of Formula II

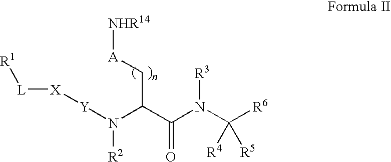

or a pharmaceutically acceptable salt thereof, where:

R¹ represents H, alkyl, alkoxy, alkenyl, alkynyl, amino, alkylamino, acylamino, cyano, sulfonylamino, acyloxy, aryl, cycloalkyl, heterocyclyl, heteroaryl, or a polypeptide chain of 1 to 8 amino acid residues;

R² represents H, lower alkyl, or aralkyl;

R³ and R⁴ independently represent H, halogen, or alkyl, or R³ and R⁴ together with the carbon to which they are attached, form a 3- to 6-membered heterocyclic ring;

R⁵ represents H, halogen, lower alkyl, or aralkyl, preferably H or lower alkyl;

R⁶ represents a functional group that reacts with an active site residue of the targeted protease to form a covalent adduct;

R⁷ represents H, aryl, alkyl, aralkyl, cycloalkyl, heterocyclyl, heteroaryl, heteroaralkyl, or a polypeptide chain of 1 to 8 amino acid residues;

R¹⁴ represents H, alkyl, alkoxy, alkenyl, alkynyl, or aralkyl, preferably H;

A is absent or represents —NHC(═NH)—, or R¹⁴ and A together with the nitrogen to which they are attached form a heterocyclic ring;

L is absent or represents alkyl, alkenyl, alkynyl, (CH₂)ₘO(CH₂)ₘ—, (CH₂)ₘNR₂(CH₂)ₘ—, and —(CH₂)ₘS(CH₂)ₘ—;

X is absent or represents —N(R⁷)—, —O—, or —S—;

Y is absent or represents —C(═O)—, —C(═S)—, or —SO₂—;

m is, independently for each occurrence, an integer from 0 to 10; and

n is an integer from 1 to 6.

In certain preferred embodiments, R¹ represents H or lower alkyl, R³ and R⁴ together with the carbon to which they are attached form a 5-membered ring, and n is an integer from 1 to 4.

In certain preferred embodiments, R¹⁴ is H, A is absent, and n is 4. In certain other embodiments R¹⁴ is H, A is —NHC(═NH)—, and n is 3.

In certain preferred embodiments, A and R¹⁴ together with the nitrogen to which they are attached form an imidazole ring, and n is 1.

In certain embodiments, R⁶ represents boronic acid, CN, —SO₂Z¹, —P(═O)Z¹, —P(═R⁸)R⁹R¹⁰, —C(═NH)NH₂, —CH═NR¹¹, or —C(═O)—R¹¹ wherein

R⁸ is O or S;

R⁹ represents N₃, SH₂, NH₂, NO₂, or OLR¹², and

R¹⁰ represents lower alkyl, amino, OLR², or a pharmaceutically acceptable salt thereof, or

R⁹ and R¹⁰, together with the phosphorus to which they are attached, form a 5- to 8-membered heterocyclic ring;

R¹¹ represents H, alkyl, alkenyl, alkynyl, NH₂, —(CH₂)ₚ—R¹², —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², (CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)NH₂, —C(O)OR¹³, or C(Z¹)(Z²)(Z³);

R¹² represents H, alkyl, alkenyl, aryl, heteroaryl, cycloalkyl, cycloalkenyl, or heterocyclyl;

R¹³ represents H, alkyl, alkenyl, or LR¹²;

Z¹ represents a halogen;

Z² and Z³ independently represent H or halogen;

p is, independently for each occurrence, an integer from 0 to 8; and

q is, independently for each occurrence, an integer from 1 to 8.

In certain preferred embodiments, R⁶ represents CN, CHO, or C(═O)C(Z¹)(Z²)(Z³), wherein Z¹ represents a halogen, and Z² and Z³ represent H or halogen. In another embodiment, R⁶ represents C(═O)C(Z¹)(Z²)(Z³), wherein Z¹ represents fluorine, and Z² and Z³ represent H or fluorine.

In certain preferred embodiments, R⁶ represents a group of formula —B(Y¹)(Y²), wherein Y¹ and Y² are independently OH or a group that is hydrolysable to OH, or together with the boron atom to which they are attached form a 5- to 8-membered ring that is hydrolysable to a boronic acid.

In certain preferred embodiments, R³ and R⁴ together with the atoms to which they are attached form a 5-membered ring, which is substituted with one or more groups selected from hydroxyl, lower alkyl (e.g., methyl), lower alkenyl, lower alkynyl, lower alkoxy, lower hydroxyalkyl (e.g., hydroxymethyl), and lower alkoxyalkyl.

In more preferred embodiments, the substituent group is selected from lower alkyl, lower hydroxyalkyl and lower alkoxyalkyl. In more preferred such embodiments, the substituent group is located at the 5-position of the ring.

In other more preferred embodiments, the substituent group is hydroxyl, which is preferably located at the 4-position of the ring.

In certain embodiments, the substituent group on the 5-membered ring containing R³ and R⁴ is selected from lower alkyl (e.g., methyl), hydroxyl, lower hydroxyalkyl (e.g., hydroxymethyl) and lower alkoxyalkyl. In certain preferred such embodiments, the substituent group has a cis-stereochemical relationship to R⁶. Such stereochemical relationships are particularly advantageous for compounds having substituents at the 4- or 5-position of the 5-membered ring, as discussed immediately above.

Exemplary structures include

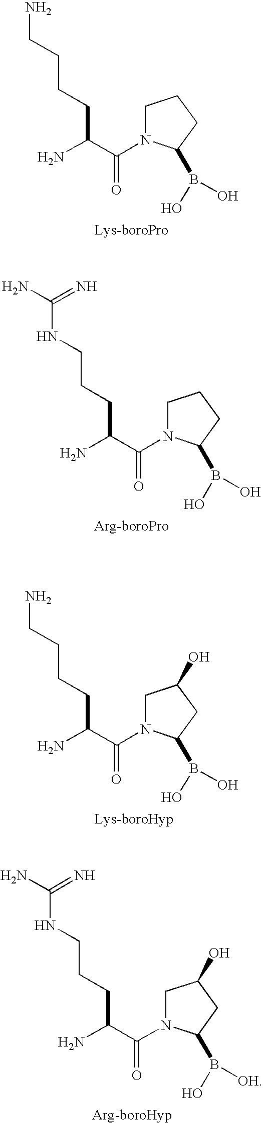

In certain embodiments of the invention, a subject compound has a structure of Formula III

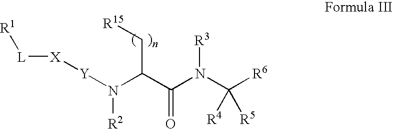

or a pharmaceutically acceptable salt thereof, where:

R¹ represents H, alkyl, alkoxy, alkenyl, alkynyl, amino, alkylamino, acylamino, cyano, sulfonylamino, acyloxy, aryl, cycloalkyl, heterocyclyl, heteroaryl, or a polypeptide chain of 1 to 8 amino acid residues;

R² represents H, lower alkyl, or aralkyl;

R³ and R⁴ independently represent H, halogen, or alkyl, or R³ and R⁴ together with the carbon to which they are attached, form a 3- to 6-membered heterocyclic ring;

R⁵ represents H, halogen, lower alkyl, or aralkyl, preferably H or lower alkyl;

R⁶ represents a functional group that reacts with an active site residue of a targeted protease to form a covalent adduct;

R⁷ represents H, aryl, alkyl, aralkyl, cycloalkyl, heterocyclyl, heteroaryl, heteroaralkyl, or a polypeptide chain of 1 to 8 amino acid residues;

R¹⁵ is a functional group that has either a positive or negative charge at physiological pH, preferably an amine or carboxylic acid;

L is absent or represents alkyl, alkenyl, alkynyl, —(CH₂)ₘO(CH₂)ₘ—, —(CH₂)ₘNR₂(CH₂)ₘ—, and —(CH₂)ₘS(CH₂)ₘ—;

X is absent or represents —N(R⁷)—, —O—, or —S—;

Y is absent or represents —C(═O)—, —C(═S)—, or —SO₂—;

m is, independently for each occurrence, an integer from 0 to 10; and

n is an integer from 1 to 6.

In certain preferred embodiments, R¹ represents H or lower alkyl, R³ is H and R⁴ is lower alkyl, or R³ and R⁴ together with the carbon to which they are attached form a 5-membered ring, and n is an integer from 1 to 4.

In certain preferred embodiments, n is an integer from 1 to 4 and R¹⁵ is a functional group that has either a positive or negative charge at physiological pH. In more preferred embodiments n is an integer from 1 to 4 and R¹⁵ is selected from amine, carboxylic acid, imidazole, or guanidine functionality.

In certain embodiments, R⁶ represents boronic acid, CN, —SO₂Z¹, —P(═O)Z¹, —P(═R⁸)R⁹R¹⁰, —C(═NH)NH₂, —CH═NR¹¹, or —C(═O)—R¹¹ wherein

R⁸ is O or S;

R⁹ represents N₃, SH₂, NH₂, NO₂, or OLR¹², and

R¹⁰ represents lower alkyl, amino, OLR¹², or a pharmaceutically acceptable salt thereof, or

R⁹ and R¹⁰, together with the phosphorus to which they are attached, form a 5- to 8-membered heterocyclic ring;

R¹¹ represents H, alkyl, alkenyl, alkynyl, NH₂, —(CH₂)ₚ—R¹², —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)NH₂, —C(O)OR¹³, or —C(Z¹)(Z²)(Z³);

R¹² represents H, alkyl, alkenyl, aryl, heteroaryl, cycloalkyl, cycloalkenyl, or heterocyclyl;

R¹³ represents H, alkyl, alkenyl, or LR¹²;

Z¹ represents a halogen;

Z² and Z³ independently represent H or halogen;

p is, independently for each occurrence, an integer from 0 to 8; and

q is, independently for each occurrence, an integer from 1 to 8.

In certain preferred embodiments, R⁶ represents CN, CHO, or C(═O)C(Z¹)(Z²)(Z³), wherein Z¹ represents a halogen, and Z² and Z³ represent H or halogen. In another embodiment, R⁶ represents C(═O)C(Z¹)(Z²)(Z³), wherein Z¹ represents fluorine, and Z² and Z³ represent H or fluorine.

In certain preferred embodiments, R⁶ represents a group of formula —B(Y¹)(Y²), wherein Y¹ and Y² are independently OH or a group that is hydrolysable to OH, or together with the boron atom to which they are attached form a 5- to 8-membered ring that is hydrolysable to a boronic acid.

In certain preferred embodiments, R³ and R⁴ together with the atoms to which they are attached form a 5-membered ring substituted with one or more groups selected from hydroxyl, lower alkyl (e.g., methyl), lower alkenyl, lower alkynyl, lower alkoxy, lower hydroxyalkyl (e.g., hydroxymethyl), and lower alkoxyalkyl.

In more preferred embodiments, the substituent group is selected from lower alkyl, lower hydroxyalkyl and lower alkoxyalkyl. In more preferred such embodiments, the substituent group is located at the 5-position of the ring.

In other more preferred embodiments, the substituent group is hydroxyl, which is preferably located at the 4-position of the ring.

In certain embodiments, the substituent group on the 5-membered ring containing R³ and R⁴ is selected from lower alkyl (e.g., methyl), hydroxyl, lower hydroxyalkyl (e.g., hydroxymethyl) and lower alkoxyalkyl. In certain preferred such embodiments, the substituent group has a cis-stereochemical relationship to R⁶. Such stereochemical relationships are particularly advantageous for compounds having substituents at the 4- or 5-position of the 5-membered ring, as discussed immediately above.

Another aspect of the invention relates to inhibitors having a structure of Formula IV:

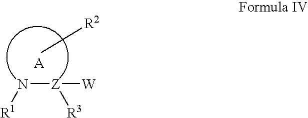

or a pharmaceutically acceptable salt thereof, wherein

A is selected from a 4-8 membered heterocycle including the N and a Cα carbon;

Z is C or N;

W is selected from CN, —CH═NR⁵, a functional group which reacts with an active site residue of the targeted protease,

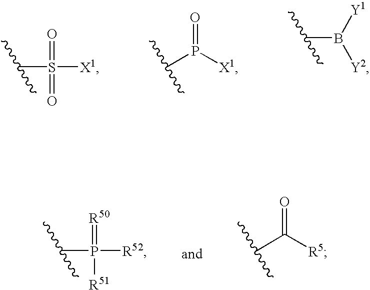

R¹ is selected from a C-terminally linked amino acid residue or amino acid analog, a C-terminally linked peptide or peptide analog, an amino-protecting group,

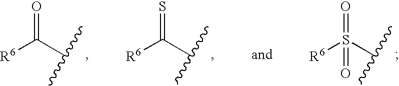

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl, thiocarbonyl, amino, acylamino, amido, cyano, nitro, azido, sulfate, sulfonate, sulfonamido, —(CH₂)ₘ—R⁷, —(CH₂)ₘ—OH, —(CH₂)ₘ—O-lower alkyl, —(CH₂)ₘ—O-lower alkenyl, —(CH₂)ₙ—O—(CH₂)ₘ—R⁷, —(CH₂)ₘ—SH, —(CH₂)ₘ—S-lower alkyl, —(CH₂)ₘ—S-lower alkenyl, or —(CH₂)ₙ—S—(CH₂)ₘ—R⁷, wherein at least one R² is selected from —OH, lower alkyl, lower alkoxy, lower hydroxyalkyl, and lower alkoxyalkyl, preferably at least one of lower alkyl (e.g., methyl), lower alkoxy, lower hydroxyalkyl (e.g., hydroxymethyl), and lower alkoxyalkyl;

when Z is N, R³ is hydrogen;

when Z is C, R³ is selected from hydrogen, halogen, lower alkyl, lower alkenyl, lower alkynyl, carbonyl, thiocarbonyl, amino, acylamino, amido, cyano, nitro, azido, sulfate, sulfonate, sulfonamido, —(CH₂)ₘ—R⁷, —(CH₂)ₘ—OH, —(CH₂)ₘ—O-lower alkyl, —(CH₂)ₘ—O-lower alkenyl, —(CH₂)ₙ—O—(CH₂)ₘ—R⁷, —(CH₂)ₘ—SH, —(CH₂)ₘ—S-lower alkyl, —(CH₂)ₘ—S-lower alkenyl, and —(CH₂)ₙ—S—(CH₂)ₘ—R⁷;

R⁵ is selected from hydrogen, alkyl, alkenyl, alkynyl, —C(X¹)(X²)X³, —(CH₂)ₘ—R⁷, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R⁷, —(CH₂)ₙ—SH, —(CH₂)ₙ—S-alkyl, —(CH₂)ₙ—S-alkenyl, —(CH₂)ₙ—S-alkynyl, —(CH₂)ₙ—S—(CH₂)ₘ—R⁷, —C(O)C(O)NH₂, and —C(O)C(O)OR^{7′};

R⁶ is selected from hydrogen, halogen, alkyl, alkenyl, alkynyl, aryl, —(CH₂)ₘ—R⁷, —(CH₂)ₘ—OH, —(CH₂)ₘ—O-alkyl, —(CH₂)ₘ—O-alkenyl, —(CH₂)ₘ—O-alkynyl, —(CH₂)ₘ—O—(CH₂)ₘ—R⁷, —(CH₂)ₘ—SH, —(CH₂)ₘ—S-alkyl, —(CH₂)ₘ—S-alkenyl, —(CH₂)ₘ—S-alkynyl, or —(CH₂)ₘ—S—(CH₂)ₘ—R⁷,

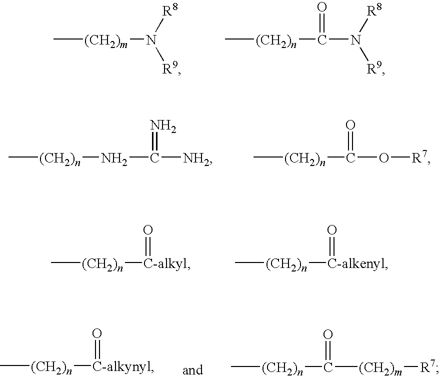

each R⁷ is independently selected from aryl, aralkyl, cycloalkyl, cycloalkenyl, and heterocyclyl;

each R^{7′} is independently selected from hydrogen, alkyl, alkenyl, aryl, aralkyl, cycloalkyl, cycloalkenyl and heterocyclyl;

R⁸ and R⁹ are each independently selected from hydrogen, alkyl, alkenyl, —(CH₂)ₘ—R⁷, —C(═O)-alkyl, —C(═O)-alkenyl, —C(═O)-alkynyl, and —C(═O)—(CH₂)ₘ—R⁷; or

R⁸ and R⁹ taken together with the N atom to which they are attached complete a heterocyclic ring having from 4 to 8 atoms in the ring structure;

R⁵⁰ is O or S;

R⁵¹ is selected from N₃, SH, NH₂, NO₂, and OR^{7′};

R⁵² is selected from hydrogen, lower alkyl, amine, OR^{7′}, or a pharmaceutically acceptable salt thereof, or

R⁵¹ and R⁵² taken together with the P atom to which they are attached complete a heterocyclic ring having from 5 to 8 atoms in the ring structure;

X¹ is a halogen;

X² and X³ are each selected from hydrogen and halogen;

Y¹ and Y² are each independently selected from OH and a group capable of being hydrolyzed to OH, including cyclic derivatives where Y¹ and Y² are connected via a ring having from 5 to 8 atoms in the ring structure;

m is zero or an integer in the range of 1 to 8; and

n is an integer in the range of 1 to 8.

In certain embodiments, W is selected from CN and B(Y¹)(Y²). In certain preferred embodiments, A is a five-membered ring, Z is C, and W is B(Y¹)(Y²). In more preferred embodiments, Z has the absolute stereochemical configuration of L-proline.

In certain embodiments, A is a five-membered ring, Z is C, and R² is selected from hydroxyl, lower alkyl, lower alkenyl, lower alkynyl, lower alkoxy, lower hydroxyalkyl, and lower alkoxyalkyl. In certain preferred such embodiments, R² is selected from lower hydroxyalkyl and lower alkoxyalkyl. In more preferred such embodiments, R² is located at the 5-position of the ring.

In certain embodiments, A is a five-membered ring, Z is C, and R² is selected from hydroxyl, lower alkyl (such as methyl), lower hydroxyalkyl (such as hydroxymethyl) and lower alkoxyalkyl. In certain preferred such embodiments, Z has the absolute stereochemical configuration of L-proline and R² is located at the 5-position of the ring for lower alkyl, lower hydroxyalkyl and lower alkoxyalkyl and at the 4-position for hydroxyl. In more preferred such embodiments, R² has a cis-stereochemical relationship to W.

Another aspect of the invention relates to inhibitors having a structure of Formula V

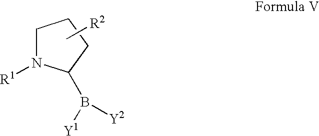

or a pharmaceutically acceptable salt thereof, wherein

R¹ is selected from a C-terminally linked amino acid residue or amino acid analog, a C-terminally linked peptide or peptide analog,

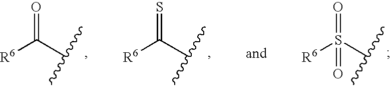

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl, thiocarbonyl, amino, acylamino, amido, cyano, nitro, azido, sulfate, sulfonate, sulfonamido, —(CH₂)ₘ—R⁷, —(CH₂)ₘ—OH, —(CH₂)ₘ—O-lower alkyl, —(CH₂)ₘ—O-lower alkenyl, —(CH₂)ₙ—O—(CH₂)ₘ—R⁷, —(CH₂)ₘ—SH, —(CH₂)ₘ—S-lower alkyl, —(CH₂)ₘ—S-lower alkenyl, or —(CH₂)ₙ—S—(CH₂)ₘ—R⁷, wherein at least one R² is selected from —OH, lower alkyl (e.g., methyl), lower alkoxy, lower hydroxyalkyl (e.g., hydroxymethyl), and lower alkoxyalkyl, preferably at least one of lower alkyl, lower alkoxy, lower hydroxyalkyl, and lower alkoxyalkyl;

R⁶ is selected from hydrogen, halogen, alkyl, alkenyl, alkynyl, aryl, —(CH₂)ₘ—R⁷, —(CH₂)ₘ—OH, —(CH₂)ₘ—O-alkyl, —(CH₂)ₘ—O-alkenyl, —(CH₂)ₘ—O-alkynyl, —(CH₂)ₘ—O—(CH₂)ₘ—R⁷, —(CH₂)ₘ—SH, —(CH₂)ₘ—S-alkyl, —(CH₂)ₘ—S-alkenyl, —(CH₂)ₘ—S-alkynyl, —(CH₂)ₘ—S—(CH₂)ₘ—R⁷,

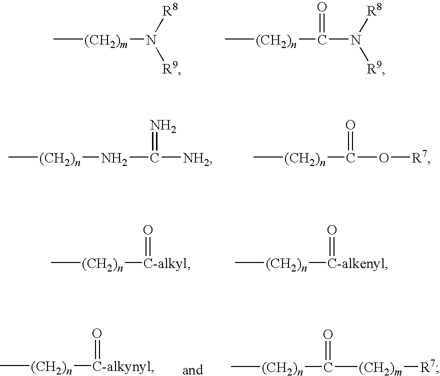

R⁷ is selected from aryl, cycloalkyl, cycloalkenyl, and heterocyclyl;

R⁸ and R⁹ are each independently selected from hydrogen, alkyl, alkenyl, —(CH₂)ₘ—R⁷, —C(═O)-alkyl, —C(═O)-alkenyl, —C(═O)-alkynyl, and —C(═O)—(CH₂)ₘ—R⁷;

or R⁸ and R⁹ taken together with the N atom to which they are attached complete a heterocyclic ring having from 4 to 8 atoms in the ring structure;

Y¹ and Y² are each independently selected from OH and a group capable of being hydrolyzed to OH, including cyclic derivatives where Y¹ and Y² are connected via a ring having from 5 to 8 atoms in the ring structure;

m is zero or an integer in the range of 1 to 8; and

n is an integer in the range of 1 to 8.

In certain embodiments, the carbon bearing B(Y¹)(Y²) has the absolute stereochemical configuration of L-proline. In certain preferred such embodiments, R² is selected from hydroxyl, lower alkyl, lower hydroxyalkyl and lower alkoxyalkyl. In more preferred such embodiments, R² is located at the 5-position of the ring for lower alkyl (such as methyl), lower hydroxyalkyl (such as hydroxymethyl) and lower alkoxyalkyl or at the 4-position for hydroxyl. In most preferred such embodiments, R² has a cis-stereochemical relationship to B(Y¹)(Y²).

Exemplary compounds include:

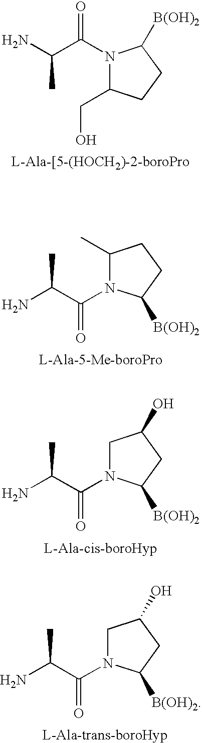

Another aspect of the invention relates to compounds having a structure of Formula VI

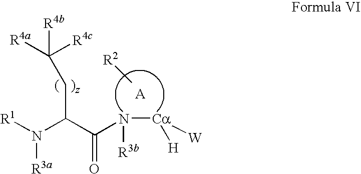

or a pharmaceutically acceptable salt thereof, wherein

A is a 3-8 membered heterocycle including the N and the Cα carbon;

W is a functional group which reacts with an active site residue of a targeted protease to form a covalent adduct;

R¹ is selected from hydrogen, a C-terminally linked amino acid or peptide or analog thereof, and an amino protecting group;

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl, thiocarbonyl, amino, acylamino, amido, cyano, nitro, azido, sulfate, sulfonate, sulfonamido, —(CH₂)ₘ—R⁶, —(CH₂)ₘ—OH, —(CH₂)ₘ—O-lower alkyl, —(CH₂)ₘ—O-lower alkenyl, —(CH₂)ₙ—O—(CH₂)ₘ—R⁶, —(CH₂)ₘ—SH, —(CH₂)ₘ—S-lower alkyl, —(CH₂)ₘ—S-lower alkenyl, and —(CH₂)ₙ—S—(CH₂)ₘ—R⁶, wherein at least one R² is selected from —OH, lower alkyl, lower alkoxy, lower hydroxyalkyl, and lower alkoxyalkyl, preferably at least one of lower alkyl (e.g., methyl), lower alkoxy (e.g., lower hydroxymethyl), lower hydroxyalkyl, and lower alkoxyalkyl;

R^{3a} is selected from hydrogen and a substituent which does not conjugate the electron pair of the nitrogen from which it pends;

R^{3b} is absent or is a substituent which does not conjugate the electron pair of the nitrogen from which it pends, such as a lower alkyl;

R^{4a} and R^{4b} are each independently selected from hydrogen, lower alkyl, heteroalkyl, cycloalkyl, heterocycloalkyl, aryl, heteroaryl, alkoxyl, carboxyl, carboxamide, carbonyl, and cyano, provided that either both or neither of R^{4a} and R^{4b} are hydrogen;

R^{4c} is selected from halogen, amine, alkyl, heteroalkyl, cycloalkyl, heterocycloalkyl, aryl, heteroaryl, alkoxyl, carboxyl, carboxamide, carbonyl, and cyano;

each R⁶ is independently selected from aryl, aralkyl, cycloalkyl, cycloalkenyl, and heterocyclyl;

z is zero or an integer in the range of 1 to 3;

m is zero or an integer in the range of 1 to 8; and

n is an integer in the range of 1 to 8.

In certain embodiments, W is selected from CN and B(Y¹)(Y²), wherein Y¹ and Y² are each independently or OH, or a group capable of being hydrolyzed to OH, including cyclic derivatives where Y¹ and Y² are connected via a ring having from 5 to 8 atoms in the ring structure. In certain preferred embodiments, A is a five-membered ring, and W is B(Y¹)(Y²). In more preferred embodiments, Cα has the absolute stereochemical configuration of L-proline.

In certain embodiments, A is a five-membered ring and R² is selected from hydroxyl, lower alkyl, lower alkenyl, lower alkynyl, lower alkoxy, lower hydroxyalkyl, and lower alkoxyalkyl. In certain preferred such embodiments, R² is selected from lower alkyl (such as methyl), lower hydroxyalkyl (such as hydroxymethyl) and lower alkoxyalkyl. In more preferred such embodiments, R² is located at the 5-position of the ring.

In certain embodiments, A is a five-membered ring, and R² is selected from hydroxyl, hydroxyl, lower alkyl, lower hydroxyalkyl and lower alkoxyalkyl. In certain preferred such embodiments, Cα has the absolute stereochemical configuration of L-proline and R² is located at the 5-position of the ring for lower alkyl (such as methyl), lower hydroxyalkyl (such as hydroxymethyl) and lower alkoxyalkyl or at the 4-position for hydroxyl. In more preferred such embodiments, R² has a cis-stereochemical relationship to W.

Another aspect of the invention relates to compounds having a structure of Formula VII:

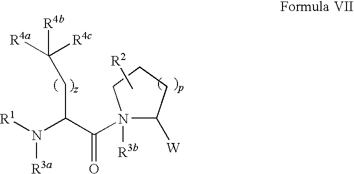

or a pharmaceutically acceptable salt thereof, wherein

R¹, R², R^{3a}, R^{3b}, R^{4a}, R^{4b}, R^{4c} and W are as defined above for Formula VI, and p is an integer from 1 to 3. In certain preferred embodiments, p is 1, and R^{3a} and R^{3b} are both hydrogen.

In certain embodiments, W is selected from CN and B(Y¹)(Y²), wherein Y¹ and Y² are each independently or OH, or a group capable of being hydrolyzed to OH, including cyclic derivatives where Y¹ and Y² are connected via a ring having from 5 to 8 atoms in the ring structure. In certain preferred embodiments, W is B(Y¹)(Y²). In more preferred embodiments, the carbon bearing W has the absolute stereochemical configuration of L-proline.

In certain embodiments, R² is selected from hydroxyl, lower alkyl, lower alkenyl, lower alkynyl, lower alkoxy, lower hydroxyalkyl, and lower alkoxyalkyl. In certain preferred embodiments, R² is selected from lower hydroxyalkyl (such as hydroxymethyl) and lower alkoxyalkyl. In more preferred such embodiments, p is 1 and R² is located at the 5-position of the ring.

In certain embodiments, R² is selected from hydroxyl, lower alkyl, lower hydroxyalkyl and lower alkoxyalkyl. In certain preferred such embodiments, p is 1, the carbon bearing W has the absolute stereochemical configuration of L-proline and R² is located at the 5-position of the ring for lower alkyl (such as methyl), lower hydroxyalkyl (such as hydroxymethyl) and lower alkoxyalkyl or at the 4-position for hydroxyl. In more preferred such embodiments, R² has a cis-stereochemical relationship to W.

Yet another aspect of the present invention relates to a compound having a structure of Formula VIII:

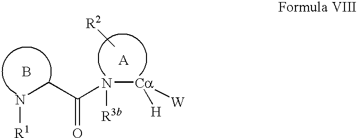

or a pharmaceutically acceptable salt thereof, wherein

A is a 3 to 8-membered heterocycle including the N and the Cα carbon;

B is a C₃₋₈ ring, or C₇₋₁₄ fused bicyclic or tricyclic ring system;

W is a functional group which reacts with an active site residue of a targeted protease to form a covalent adduct, as for example, —CN, —CH═NR⁵,

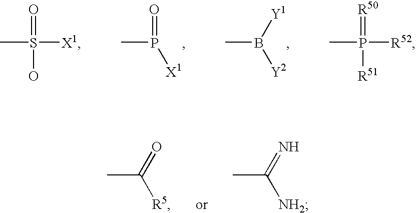

R¹ is selected from hydrogen, a C-terminally linked amino acid or peptide or analog thereof, and an amino protecting group;

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl (such as carboxyl, ester, formate, or ketone), thiocarbonyl (such as thioester, thioacetate, or thioformate), amino, acylamino, amido, cyano, nitro, azido, sulfate, sulfonate, sulfonamido, —(CH₂)ₘ—R⁶, —(CH₂)ₘ—OH, —(CH₂)ₘ—O-lower alkyl, —(CH₂)ₘ—O-lower alkenyl, —(CH₂)ₙ—O—(CH₂)ₘ—R⁶, (CH₂)ₘ—SH, (CH₂)ₘ—S-lower alkyl, —(CH₂)ₘ—S-lower alkenyl, and —(CH₂)ₙ—S—(CH₂)ₘ—R⁶, wherein at least one R² is selected from —OH, lower alkyl, lower alkoxy, lower hydroxyalkyl, and lower alkoxyalkyl, preferably at least one of lower alkyl (such as methyl), lower alkoxy (such as hydroxymethyl), lower hydroxyalkyl, and lower alkoxyalkyl;

R^{3b} is absent, or represents a substituent which does not conjugate the electron pair of the nitrogen from which it pends, such as a lower alkyl;

R⁵ is selected from hydrogen, alkyl, alkenyl, alkynyl, —C(X¹)(X²)X³, —(CH₂)ₘ—R⁶, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R⁶, —(CH₂)ₙ—SH, —(CH₂)ₙ—S-alkyl, —(CH₂)ₙ—S-alkenyl, —(CH₂)ₙ—S-alkynyl, —(CH₂)ₙ—S—(CH₂)ₘ—R⁶, —C(O)C(O)NH₂, and —C(O)C(O)OR⁷;

each R⁶ is independently selected from aryl, aralkyl, cycloalkyl, cycloalkenyl, and heterocyclyl;

each R⁷ is independently selected from hydrogen, alkyl, alkenyl, aryl, aralkyl, cycloalkyl, cycloalkenyl, and heterocycle;

Y¹ and Y² are each independently selected from —OH, or a group capable of being hydrolyzed to a hydroxyl group, including cyclic derivatives where Y¹ and Y² are connected via a ring having from 5 to 8 atoms in the ring structure (such as pinacol or the like),

R⁵⁰ is O or S;

R⁵¹ is selected from N₃, SH₂, NH₂, NO₂ or —OR⁷;

R⁵² represents hydrogen, a lower alkyl, an amine, —OR⁷, or a pharmaceutically acceptable salt thereof, or R⁵¹ and R⁵² taken together with the phosphorous atom to which they are attached complete a heterocyclic ring having from 5 to 8 atoms in the ring structure

X¹ represents a halogen;

X² and X³ are each independently selected from hydrogen and halogen;

m is zero or an integer in the range of 1 to 8; and

n is an integer in the range of 1 to 8.

In certain embodiments, W is selected from CN and B(Y¹)(Y²), wherein Y¹ and Y² are each independently or OH, or a group capable of being hydrolyzed to OH, including cyclic derivatives where Y¹ and Y² are connected via a ring having from 5 to 8 atoms in the ring structure. In certain preferred embodiments, A is a five-membered ring, and W is B(Y¹)(Y²). In more preferred embodiments, Ca has the absolute stereochemical configuration of L-proline.

In certain embodiments, A is a five-membered ring and R² is selected from hydroxyl, lower alkyl, lower alkenyl, lower alkynyl, lower alkoxy, lower hydroxyalkyl, and lower alkoxyalkyl. In certain preferred such embodiments, R² is selected from lower hydroxyalkyl (hydroxymethyl) and lower alkoxyalkyl. In more preferred such embodiments, R² is located at the 5-position of the ring.

In certain embodiments, A is a five-membered ring, and R² is selected from hydroxyl, lower alkyl, lower hydroxyalkyl and lower alkoxyalkyl. In certain preferred such embodiments, Cα has the absolute stereochemical configuration of L-proline and R² is located at the 5-position of the ring for lower alkyl (such as methyl), lower hydroxyalkyl (such as hydroxymethyl) and lower alkoxyalkyl or at the 4-position for hydroxyl. In more preferred such embodiments, R² has a cis-stereochemical relationship to W.

Another aspect of the invention relates to compounds having a structure of Formula IX:

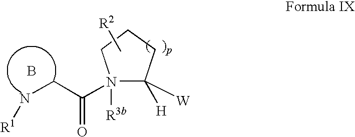

or a pharmaceutically acceptable salt thereof, wherein

B, R¹, R², R^{3b} and W are as defined above for Formula VIII, and p is an integer from 1 to 3. In certain preferred embodiments, p is 1, and R^{3b} is hydrogen.

In certain embodiments, W is selected from CN and B(Y¹)(Y²), wherein Y¹ and Y² are each independently or OH, or a group capable of being hydrolyzed to OH, including cyclic derivatives where Y¹ and Y² are connected via a ring having from 5 to 8 atoms in the ring structure. In certain preferred embodiments, W is B(Y¹)(Y²). In more preferred embodiments, the carbon bearing W has the absolute stereochemical configuration of L-proline.

In certain embodiments, R² is selected from hydroxyl, lower alkyl, lower alkenyl, lower alkynyl, lower alkoxy, lower hydroxyalkyl, and lower alkoxyalkyl. In certain preferred such embodiments, R² is selected from lower hydroxyalkyl (such as hydroxymethyl) and lower alkoxyalkyl. In more preferred such embodiments, R² is located at the 5-position of the ring.

In certain embodiments, R² is selected from hydroxyl, lower alkyl, lower hydroxyalkyl and lower alkoxyalkyl. In certain preferred such embodiments, p is 1, the carbon bearing W has the absolute stereochemical configuration of L-proline and R² is located at the 4-position of the ring for hydroxyl or at the 5-position for lower alkyl (such as methyl), lower hydroxyalkyl (such as hydroxymethyl) and lower alkoxyalkyl. In more preferred such embodiments, R² has a cis-stereochemical relationship to W.

Another aspect of the invention relates to compounds having a structure of Formula X

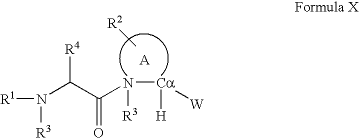

or a pharmaceutically acceptable salt thereof, wherein

A is a 4-8 membered heterocycle including the N and the Cα carbon;

W is a functional group which reacts with an active site residue of the targeted protease to form a covalent adduct, as for example, —CN, —CH═NR₅,

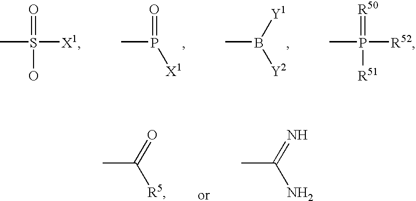

R¹ represents a C-terminally linked peptide or peptide analog which is a substrate for an activating enzyme;

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl, thiocarbonyl, amino, acylamino, amido, cyano, nitro, azido, sulfate, sulfonate, sulfonamido, —(CH₂)ₘ—R⁶, —(CH₂)ₘ—OH, —(CH₂)ₘ—O-lower alkyl, —(CH₂)ₘ—O-lower alkenyl, —(CH₂)ₙ—O—(CH₂)ₘ—R⁶, —(CH₂)ₘ—SH, —(CH₂)ₘ—S-lower alkyl, —(CH₂)ₘ—S-lower alkenyl, —(CH₂)ₙ—S—(CH₂)ₘ—R⁶, wherein at least one R² is selected from —OH, lower alkyl, lower alkoxy, lower hydroxyalkyl, and lower alkoxyalkyl, preferably at least one of lower alkyl (e.g., methyl), lower alkoxy (e.g., hydroxymethyl), lower hydroxyalkyl, and lower alkoxyalkyl;

each R³ is independently selected from hydrogen and a substituent which does not conjugate the electron pair of the nitrogen from which it pends, such as a lower alkyl;

R⁴ is selected from hydrogen and a small hydrophobic group such as a halogen, lower alkyl, lower alkenyl, or lower alkynyl;

R⁵ is selected from hydrogen, alkyl, alkenyl, alkynyl, —C(X¹)(X²)X³, —(CH²)^{m}—R⁶, —(CH²)ⁿ—OH, (CH²)ⁿ—O-alkyl, —(CH²)ⁿ—O-alkenyl, —(CH²)ⁿ-alkynyl, —(CH²)ⁿ—O—(CH²)^{m}—R⁶, —(CH²)ⁿ—SH, —(CH²)ⁿS-alkyl, —(CH²)ⁿ—S-alkenyl, —(CH²)ⁿ—S-alkynyl, —(CH²)ⁿ—S—(CH²)^{m}—R⁶, —C(O)C(O)NH², —C(O)C(O)OR⁷;

R⁶ represents, for each occurrence, a substituted or unsubstituted aryl, aralkyl, cycloalkyl, cycloalkenyl, or heterocycle;

R⁷ represents, for each occurrence, hydrogen, or a substituted or unsubstituted alkyl, alkenyl, aryl, aralkyl, cycloalkyl, cycloalkenyl, or heterocycle; and

Y¹ and Y² are independently or together OH or a group capable of being hydrolyzed to a hydroxyl group, including cyclic derivatives where Y¹ and Y² are connected via a ring having from 5 to 8 atoms in the ring structure (such as pinacol or the like),

R⁵⁰ is O or S;

R⁵¹ is selected from N₃, SH₂, NH₂, NO₂ and —OR⁷;

R⁵² is selected from hydrogen, lower alkyl, amine, —OR⁷, or a pharmaceutically acceptable salt thereof; or

R⁵¹ and R⁵² taken together with the phosphorous atom to which they are attached complete a heterocyclic ring having from 5 to 8 atoms in the ring structure

X¹ is a halogen;

X² and X³ are each independently selected from hydrogen and halogen;

m is zero or an integer in the range of 1 to 8; and

n is an integer in the range of 1 to 8.

In certain embodiments, W is selected from CN and B(Y¹)(Y²). In certain preferred embodiments, A is a five-membered ring, and W is B(Y¹)(Y²). In more preferred embodiments, Cα has the absolute stereochemical configuration of L-proline.

In certain embodiments, A is a five-membered ring, Z is C, and R² is selected from hydroxyl, lower alkyl, lower alkenyl, lower alkynyl, lower alkoxy, lower hydroxyalkyl, and lower alkoxyalkyl. In certain preferred such embodiments, R² is selected from lower hydroxyalkyl (such as hydroxymethyl) and lower alkoxyalkyl. In more preferred such embodiments, R² is located at the 5-position of the ring.

In certain embodiments, A is a five-membered ring and R² is selected from hydroxyl, lower alkyl, lower hydroxyalkyl and lower alkoxyalkyl. In certain preferred such embodiments, Cα has the absolute stereochemical configuration of L-proline and R² is located at the 4-position of the ring for hydroxyl or at the 5-position for lower alkyl, lower hydroxyalkyl and lower alkoxyalkyl. In more preferred such embodiments, R² has a cis-stereochemical relationship to W.

One aspect of the invention relates to compounds having a structure of Formula XI

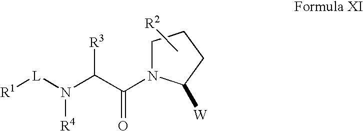

or a pharmaceutically acceptable salt thereof, wherein

L is absent or is —XC(O)—;

R¹ is selected from H, lower alkyl, lower acyl, lower aralkyl, lower aracyl, lower heteroaracyl, carbocyclyl, aryl, and ArSO₂—;

R² represents one or more substitutions to the ring A, each of which is independently selected from halogen, lower alkyl, lower alkenyl, lower alkynyl, lower alkoxy, lower hydroxyalkyl, lower alkoxyalkyl, carbonyl, thiocarbonyl, amino, acylamino, amido, cyano, nitro, azido, sulfate, sulfonate, sulfonamido, —(CH₂)ₘ—R⁶, —(CH₂)ₘ—OH, —(CH₂)ₘ—O-lower alkyl, —(CH₂)ₘ—O-lower alkenyl, —(CH₂)ₙ—O—(CH₂)ₘ—R⁶, —(CH₂)ₘ—SH, —(CH₂)ₘ—S-lower alkyl, —(CH₂)ₘ—S-lower alkenyl, —(CH₂)ₙ—S—(CH₂)ₘ—R⁶, wherein at least one R² is selected from —OH, lower alkyl, lower alkoxy, lower hydroxyalkyl, and lower alkoxyalkyl, preferably at least one of lower alkyl (e.g., methyl), lower alkoxy (e.g., hydroxymethyl), lower hydroxyalkyl, and lower alkoxyalkyl;

R³ is selected from hydrogen, lower alkyl, lower hydroxyalkyl, lower thioalkyl, and lower aralkyl;

R⁴ is selected from H and lower alkyl, or R¹ and R⁴ together are phthaloyl, thereby forming a ring;

R⁶ represents, for each occurrence, a substituted or unsubstituted aryl, aralkyl, cycloalkyl, cycloalkenyl, or heterocycle;

W is selected from B(Y¹)(Y²) and CN;

Y¹ and Y² are independently selected from OH or a group that is hydrolyzable to an OH, or together with the boron atom to which they are attached form a 5- to 8-membered ring that is hydrolysable to OH;

X is selected from O and NH.

In certain embodiments, W is B(Y¹)(Y²). In certain preferred embodiments, R² is selected from hydroxyl, lower alkyl, lower alkenyl, lower alkynyl, lower alkoxy, lower hydroxyalkyl, and lower alkoxyalkyl. In more preferred such embodiments, R² is selected from lower hydroxyalkyl and lower alkoxyalkyl. In more preferred such embodiments, R² is located at the 5-position of the ring.

In certain embodiments, R² is selected from hydroxyl, lower alkyl, lower hydroxyalkyl and lower alkoxyalkyl. In certain preferred such embodiments, Cα has the absolute stereochemical configuration of L-proline and R² is located at the 4-position of the ring for hydroxyl or at the 5-position for lower alkyl, lower hydroxyalkyl and lower alkoxyalkyl. In more preferred such embodiments, R² has a cis-stereochemical relationship to W.

In certain preferred embodiments, the subject inhibitors are DPIV inhibitors with a Kᵢ for DPIV inhibition of 10 nm or less, more preferably of 1.0 nm or less, and even more preferably of 0.1 or even 0.01 nM or less. Indeed, inhibitors with Kᵢ values in the picomolar and even femtomolar range are contemplated.

In general, the inhibitors of the subject method are small molecules, e.g., with molecular weights less than 7500 amu, preferably less than 5000 amu, and even more preferably less than 2000 amu and even less than 1000 amu. In preferred embodiments, the inhibitors are orally active.

Another aspect of the present invention relates to pharmaceutical compositions of dipeptidylpeptidase inhibitors, particularly inhibitor(s) and their uses in treating and/or preventing disorders which can be improved by altering the homeostasis of peptide hormones. In a preferred embodiment, the inhibitors have hypoglycemic and antidiabetic activities, and can be used in the treatment of disorders marked by aberrant glucose metabolism (including storage). In particular embodiments, the compositions of the subject methods are useful as insulinotropic agents, or to potentiate the insulinotropic effects of such molecules as GLP-1. In this regard, the present method can be useful for the treatment and/or prophylaxis of a variety of disorders, including one or more of: hyperlipemia, hyperglycemia, obesity, glucose tolerance insufficiency, insulin resistance, and diabetic complications.

For instance, in certain embodiments the method involves administration of an inhibitor(s), preferably at a predetermined interval(s) during a 24-hour period, in an amount effective to improve one or more aberrant indices associated with glucose metabolism disorders (e.g., glucose intolerance, insulin resistance, hyperglycemia, hyperinsulinemia, and Type II diabetes). The effective amount of the inhibitor may be about 0.01, 0.1, 1, 10, 30, 50, 70, 100, 150, 200, 500, or 1000 mg/kg of the subject.

(ii). Agonism of GLP-1 Effects

The inhibitors useful in the subject methods possess, in certain embodiments, the ability to lower blood glucose levels, to relieve obesity, to alleviate impaired glucose tolerance, to inhibit hepatic glucose neogenesis, and to lower blood lipid levels and to inhibit aldose reductase. They are thus useful for the prevention and/or therapy of hyperglycemia, obesity, hyperlipidemia, diabetic complications (including retinopathy, nephropathy, neuropathy, cataracts, coronary artery disease and arteriosclerosis), and furthermore for obesity-related hypertension and osteoporosis.

Diabetes mellitus is a disease characterized by hyperglycemia occurring from a relative or absolute decrease in insulin secretion, decreased insulin sensitivity, or insulin resistance. The morbidity and mortality of this disease result from vascular, renal, and neurological complications. An oral glucose tolerance test is a clinical test used to diagnose diabetes. In an oral glucose tolerance test, a patient's physiological response to a glucose load or challenge is evaluated. After ingesting the glucose, the patient's physiological response to the glucose challenge is evaluated. Generally, this is accomplished by determining the patient's blood glucose levels (the concentration of glucose in the patient's plasma, serum, or whole blood) for several predetermined points in time.

In one embodiment, the present invention provides a method for agonizing the action of GLP-1. It has been determined that isoforms of GLP-1 (GLP-1(7-37) and GLP-1(7-36)), which are derived from preproglucagon in the intestine and the hind brain, have insulinotropic activity, i.e., they modulate glucose metabolism. DPIV cleaves the isoforms to inactive peptides. Thus, in certain embodiments, inhibitor(s) of the present invention can agonize insulinotropic activity by interfering with the degradation of bioactive GLP-1 peptides.

(iii). Agonism of the Effects of Other Peptide Hormones

In another embodiment, the subject agents can be used to agonize (e.g., mimic or potentiate) the activity of peptide hormones, e.g., GLP-2, GIP and NPY.

To illustrate further, the present invention provides a method for agonizing the action of GLP-2. It has been determined that GLP-2 acts as a trophic agent, to promote growth of gastrointestinal tissue. The effect of GLP-2 is marked particularly by increased growth of the small bowel, and is therefore herein referred to as an “intestinotrophic” effect. DPIV is known to cleave GLP-2 into a biologically inactive peptide. Thus, in one embodiment, inhibition of DPIV interferes with the degradation of GLP-2, and thereby increases the plasma half-life of that hormone.

In still other embodiments, the subject method can be used to increase the half-life of other proglucagon-derived peptides, such as glicentin, oxyntomodulin, glicentin-related pancreatic polypeptide (GRPP), and/or intervening peptide-2 (IP-2). For example, glicentin has been demonstrated to cause proliferation of intestinal mucosa and also inhibits a peristalsis of the stomach, and has thus been elucidated as useful as a therapeutic agent for digestive tract diseases, thus leading to the present invention.

Thus, in one aspect, the present invention relates to therapeutic and related uses of inhibitor(s) for promoting the growth and proliferation of gastrointestinal tissue, most particularly small bowel tissue. For instance, the subject method can be used as part of a regimen for treating injury, inflammation, or resection of intestinal tissue, e.g., where enhanced growth and repair of the intestinal mucosal epithelial is desired.

With respect to small bowel tissue, such growth is measured conveniently as an increase in small bowel mass and length, relative to an untreated control. The effect of subject inhibitors on small bowel also manifests as an increase in the height of the crypt plus villus axis. Such activity is referred to herein as an “intestinotrophic” activity. The efficacy of the subject method may also be detectable as an increase in crypt cell proliferation and/or a decrease in small bowel epithelium apoptosis. These cellular effects may be noted most significantly in relation to the jejunum, including the distal jejunum and particularly the proximal jejunum, and also in the distal ileum. A compound is considered to have “intestinotrophic effect” if a test animal exhibits significantly increased small bowel weight, increased height of the crypt plus villus axis or increased crypt cell proliferation, or decreased small bowel epithelium apoptosis when treated with the compound (or genetically engineered to express it themselves). A model suitable for determining such gastrointestinal growth is described by U.S. Pat. No. 5,834,428.

In general, patients who would benefit from either increased small intestinal mass and consequent increased small bowel mucosal function are candidates for treatment by the subject method. Particular conditions that may be treated include the various forms of sprue, including celiac sprue which results from a toxic reaction to α-gliadin from wheat, and is marked by a tremendous loss of villae of the bowel; tropical sprue which results from infection and is marked by partial flattening of the villae; hypogammaglobulinemic sprue which is observed commonly in patients with common variable immunodeficiency or hypogammaglobulinemia and is marked by significant decrease in villus height. The therapeutic efficacy of the treatment may be monitored by enteric biopsy to examine the villus morphology, by biochemical assessment of nutrient absorption, by patient weight gain, or by amelioration of the symptoms associated with these conditions. Other conditions that may be treated by the subject method, or for which the subject method may be useful prophylactically, include radiation enteritis, infectious or post-infectious enteritis, regional enteritis (Crohn's disease), small intestinal damage due to toxic or other chemotherapeutic agents, and patients with short bowel syndrome.

More generally, the present invention provides a therapeutic method for treating digestive tract diseases. The term “digestive tract” as used herein means a tube through which food passes, including stomach and intestine. The term “digestive tract diseases” as used herein means diseases accompanied by a qualitative or quantitative abnormality in the digestive tract mucosa, which include, e.g., ulceric or inflammatory disease; congenital or acquired digestion and absorption disorder including malabsorption syndrome; disease caused by loss of a mucosal barrier function of the gut; and protein-losing gastroenteropathy. The ulceric disease includes, e.g., gastric ulcer, duodenal ulcer, small intestinal ulcer, colonic ulcer, and rectal ulcer. The inflammatory disease include, e.g., esophagitis, gastritis, duodenitis, enteritis, colitis, Crohn's disease, proctitis, gastrointestinal Behcet, radiation enteritis, radiation colitis, radiation proctitis, enteritis, and medicamentosa. The malabsorption syndrome includes the essential malabsorption syndrome such as disaccharide-decomposing enzyme deficiency, glucose-galactose malabsorption, fructose malabsorption; secondary malabsorption syndrome, e.g., the disorder caused by a mucosal atrophy in the digestive tract through the intravenous or parenteral nutrition or elemental diet, the disease caused by the resection and shunt of the small intestine such as short gut syndrome, cul-de-sac syndrome; and indigestible malabsorption syndrome, such as the disease caused by resection of the stomach, e.g., dumping syndrome.

The term “therapeutic agent for digestive tract diseases” as used herein means the agents for the prevention and treatment of the digestive tract diseases, which include, e.g., the therapeutic agent for digestive tract ulcer, the therapeutic agent for inflammatory digestive tract disease, the therapeutic agent for mucosal atrophy in the digestive tract, the therapeutic agent for a digestive tract wound, the amelioration agent for the function of the digestive tract including the agent for recovery of the mucosal barrier function, and the amelioration agent for digestive and absorptive function. Ulcers include digestive ulcers and erosions, and acute ulcers, namely acute mucosal lesions.

The subject method, because of promoting proliferation of intestinal mucosa, can be used in the treatment and prevention of pathologic conditions of insufficiency in digestion and absorption, that is, treatment and prevention of mucosal atrophy, or treatment of hypoplasia of the digestive tract tissues and decrease in these tissues by surgical removal as well as improvement of digestion and absorption. Further, the subject method can be used in the treatment of pathologic mucosal conditions due to inflammatory diseases such as enteritis, Crohn's disease, and ulceric colitis and also in the treatment of reduction in function of the digestive tract after operation, for example, in damping syndrome as well as in the treatment of duodenal ulcer in conjunction with the inhibition of peristalsis of the stomach and rapid migration of food from the stomach to the jejunum. Furthermore, glicentin can effectively be used in promoting cure of surgical invasion as well as in improving functions of the digestive tract. Thus, the present invention also provides a therapeutic agent for atrophy of the digestive tract mucosa, a therapeutic agent for wounds in the digestive tract and a drug for improving functions of the digestive tract which comprise glicentin as active ingredients.

Likewise, the inhibitor(s) of the subject invention can be used to alter the plasma half-life of secretin, VIP, PHI, PACAP, GIP, and/or helodermin. Additionally, the subject method can be used to alter the pharmacokinetics of Peptide YY and neuropeptide Y, both members of the pancreatic polypeptide family, as DPIV has been implicated in the processing of those peptides in a manner which alters receptor selectivity.

Neuropeptide Y (NPY) is believed to act in the regulation vascular smooth muscle tone, as well as regulation of blood pressure. NPY also decreases cardiac contractility. NPY is also the most powerful appetite stimulant known (Wilding et al., (1992) J Endocrinology 132:299-302). The centrally evoked food intake (appetite stimulation) effect is predominantly mediated by NPY Y1 receptors and causes increase in body fat stores and obesity (Stanley et al., (1989) Physiology and Behavior 46:173-177).

According to the present invention, a method for treatment of anorexia comprises administering to a host subject an effective amount of an inhibitor(s) to stimulate the appetite and increase body fat stores which thereby substantially relieves the symptoms of anorexia.

A method for treatment of hypotension comprises administering to a host subject an effective amount of an inhibitor(s) of the present invention to mediate vasoconstriction and increase blood pressure which thereby substantially relieves the symptoms of hypotension.

DPIV has also been implicated in the metabolism and inactivation of growth hormone-releasing factor (GHRF). GHRF is a member of the family of homologous peptides that includes glucagon, secretin, vasoactive intestinal peptide (VIP), peptide histidine isoleucine (PHI), pituitary adenylate cyclase activating peptide (PACAP), gastric inhibitory peptide (GIP) and helodermin (Kubiak et al. (1994) Peptide Res 7:153). GHRF is secreted by the hypothalamus, and stimulates the release of growth hormone (GH) from the anterior pituitary. Thus, the subject method can be used to improve clinical therapy for certain growth hormone deficient children, and in clinical therapy of adults to improve nutrition and to alter body composition (muscle vs. fat). The subject method can also be used in veterinary practice, for example, to develop higher yield milk production and higher yield, leaner livestock.

(iv). Assays of Insulinotropic Activity

In selecting a compound suitable for use in the subject method, it is noted that the insulinotropic property of a compound may be determined by providing that compound to animal cells, or injecting that compound into animals and monitoring the release of immunoreactive insulin (IRI) into the media or circulatory system of the animal, respectively. The presence of IRI can be detected through the use of a radioimmunoassay which can specifically detect insulin.

The db/db mouse is a genetically obese and diabetic strain of mouse. The db/db mouse develops hyperglycemia and hyperinsulinemia concomitant with its development of obesity and thus serves as a model of obese type 2 diabetes (NIDDM). The db/db mice can be purchased from, for example, The Jackson Laboratories (Bar Harbor, Me.). In an exemplary embodiment, for treatment of the mice with a regimen including an inhibitor(s) or control, sub-orbital sinus blood samples are taken before and at some time (e.g., 60 minutes) after dosing of each animal. Blood glucose measurements can be made by any of several conventional techniques, such as using a glucose meter. The blood glucose levels of the control and inhibitor(s) dosed animals are compared

The metabolic fate of exogenous GLP-1 can also be followed in both nondiabetic or type II diabetic subjects, and the effect of a candidate inhibitor(s) determined. For instance, a combination of high-pressure liquid chromatography (HPLC), specific radioimmunoassays (RIAs), and an enzyme-linked immunosorbent assay (ELISA), can be used, whereby intact biologically active GLP-1 and its metabolites can be detected. See, for example, Deacon et al. (1995) Diabetes 44:1126-1131. To illustrate, after GLP-1 administration, the intact peptide can be measured using an NH₂-terminally directed RIA or ELISA, while the difference in concentration between these assays and a COOH-terminal-specific RIA allowed determination of NH₂-terminally truncated metabolites. Without inhibitor, subcutaneous GLP-1 is rapidly degraded in a time-dependent manner, forming a metabolite which co-elutes on HPLC with GLP-I(9-36) amide and has the same immunoreactive profile. For instance, thirty minutes after subcutaneous GLP-1 administration to diabetic patients (n=8), the metabolite accounted for 88.5+1.9% of the increase in plasma immunoreactivity determined by the COOH-terminal RIA, which was higher than the levels measured in healthy subjects (78.4+3.2%; n=8; P<0.05). See Deacon et al., supra. Intravenously infused GLP-I was also extensively degraded.

(v). Conjoint Administration

Another aspect of the invention provides a conjoint therapy wherein one or more other therapeutic agents are administered with the protease inhibitor. Such conjoint treatment may be achieved by way of the simultaneous, sequential, or separate dosing of the individual components of the treatment.

In one embodiment, an inhibitor(s) is conjointly administered with insulin or other insulinotropic agents, such as GLP-1, peptide hormones, such as GLP-2, GIP, or NPY, or a gene therapy vector which causes the ectopic expression of said agents and peptide hormones. In certain embodiments, said agents or peptide hormones may be variants of a naturally occurring or synthetic peptide hormone, wherein one or more amino acids have been added, deleted, or substituted.

In another illustrative embodiment, the subject inhibitors can be conjointly administered with an M1 receptor antagonist. Cholinergic agents are potent modulators of insulin release that act via muscarinic receptors. Moreover, the use of such agents can have the added benefit of decreasing cholesterol levels, while increasing HDL levels. Suitable muscarinic receptor antagonists include substances that directly or indirectly block activation of muscarinic cholinergic receptors. Preferably, such substances are selective (or are used in amounts that promote such selectivity) for the M1 receptor. Nonlimiting examples include quaternary amines (such as methantheline, ipratropium, and propantheline), tertiary amines (e.g. dicyclomine and scopolamine), and tricyclic amines (e.g. telenzepine). Pirenzepine and methyl scopolamine are preferred. Other suitable muscarinic receptor antagonists include benztropine (commercially available as COGENTIN from Merck), hexahydro-sila-difenidol hydrochloride (HHSID hydrochloride disclosed in Lambrecht et al. (1989) Trends in Pharmacol. Sci. 10(Suppl):60; (+/−)-3-quinuclidinyl xanthene-9-carboxylate hemioxalate (QNX-hemioxalate; Birdsall et al., Trends in Pharmacol. Sci. 4:459, 1983; telenzepine dihydrochloride (Coruzzi et al. (1989) Arch. Int. Pharmacodyn. Ther. 302:232; and Kawashima et al. (1990) Gen. Pharmacol. 21:17), and atropine. The dosages of such muscarinic receptor antagonists will be generally subject to optimization as outlined above. In the case of lipid metabolism disorders, dosage optimization may be necessary independent of whether administration is timed by reference to the lipid metabolism responsiveness window or not.

In terms of regulating insulin and lipid metabolism and reducing the foregoing disorders, the subject inhibitor(s) may also act synergistically with prolactin inhibitors such as d2 dopamine agonists (e.g. bromocriptine). Accordingly, the subject method can include the conjoint administration of such prolactin inhibitors as prolactin-inhibiting ergo alkaloids and prolactin-inhibiting dopamine agonists. Examples of suitable compounds include 2-bromo-alpha-ergocriptine, 6-methyl-8 beta-carbobenzyloxyaminoethyl-10-alpha-ergoline, 8-acylaminoergolines, 6-methyl-8-alpha-(N-acyl)amino-9-ergoline, 6-methyl-8-alpha-(N-phenylacetyl)amino-9-ergoline, ergocornine, 9,10-dihydroergocornine, D-2-halo-6-alkyl-8-substituted ergolines, D-2-bromo-6-methyl-8-cyanomethylergoline, carbidopa, benserazide, and other dopadecarboxylase inhibitors, L-dopa, dopamine, and non toxic salts thereof.

The inhibitor(s) used according to the invention can also be used conjointly with agents acting on the ATP-dependent potassium channel of the β-cells, such as glibenclamide, glipizide, gliclazide, and AG-EE 623 ZW. The inhibitor(s) may also advantageously be applied in combination with other oral agents such as metformin and related compounds or glucosidase inhibitors as, for example, acarbose.

(vi). Pharmaceutical Compositions

Inhibitors prepared as described herein can be administered in various forms, depending on the disorder to be treated and the age, condition, and body weight of the patient, as is well known in the art. For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means, and, if desired, the active ingredient may be mixed with any conventional additive, such as an excipient, a binder, a disintegrating agent, a lubricant, a corrigent, a solubilizing agent, a suspension aid, an emulsifying agent, or a coating agent. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses.

The precise time of administration and/or amount of the inhibitor that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19)

In other cases, the inhibitors useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an inhibitor(s) with the carrier and, optionally, one or more accessory ingredients. In general, the formulations are prepared by uniformly and intimately bringing into association a ligand with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes, and the like, each containing a predetermined amount of an inhibitor(s) as an active ingredient. A compound may also be administered as a bolus, electuary or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to inhibitor(s), excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an inhibitor(s), excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

The inhibitor(s) can be alternatively administered by aerosol. This is accomplished by preparing an aqueous aerosol, liposomal preparation, or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of an inhibitor(s) to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the peptidomimetic in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more inhibitors(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the inhibitors(s) of the present invention are administered as pharmaceuticals to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of agents may be given orally, parenterally, topically, or rectally. They are of course given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

These inhibitors(s) may be administered to humans and other animals for therapy by any suitable route of administration, including orally, nasally, as by, for example, a spray, rectally, intravaginally, parenterally, intracistemally, and topically, as by powders, ointments or drops, including buccally and sublingually.

Regardless of the route of administration selected, the inhibitor(s), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

### IV. Exemplification

The invention now being generally described, it will be more readily understood by reference to the following examples which are included merely for purposes of illustration of certain aspects and embodiments of the present invention, and are not intended to limit the invention.

### Example 1

### DPIV Inhibition Assay

The inhibitor solution was prepared by dissolving 3-5 mg of inhibitor in pH 2 solution (0.01 N HCl), such that the concentration of the solution was equal to 1 mg/10 μL. A 10 μL sample of this solution was then added to 990 μL of pH 8 buffer (0.1 M HEPES, 0.14 M NaCl), and the solution was allowed to stand at room temperature overnight.

The enzyme solution was prepared by diluting 20 μL of DPIV (concentration 2.5 μM) into 40 mL of pH 8 buffer.

The substrate solution was prepared by dissolving 2.0 mg of L-alanyl-L-proline-para-nitroanilide into 20 mL of pH 8 buffer. 250 μL of enzyme solution was added to well #B1 to #H1, #A2 to #H2, and #A3 to #H3 of a 96 well plate, while well #A1 received 250 μL of pH 8 buffer instead of enzyme solution. 90 μL of pH 8 buffer was then added to column 5 (from well #A5 to #H5).

A 1:10 dilution was then performed by adding inhibitor solution to #A5 and the solution was mixed well before transferring 10 μL of this solution from #A5 to #B5. The solution in #B5 was then mixed well before transferring 10 μL of this solution from #B5 to #C5. The solution in #C5 was then mixed well before transferring 10 μL of this solution from #C5 to #D5. The solution in #D5 was then mixed well before transferring 10 μL of this solution from #D5 to #E5. The solution in #E5 was then mixed well before transferring 10 μL of this solution from #E5 to #F5. The solution in #F5 was then mixed well before transferring 10 μL of this solution from #F5 to #G5. The solution in #G5 was then mixed well before transferring 10 μL of this solution from #G5 to #H5.

A 30 μL aliquot was then transferred from #H5 to #H3 for row H, and the contents were mixed well. The analogous procedure was repeated for rows G, F, E, D, C, B, and A sequentially. The plate was then shaken on a plate shaker for 5 minutes before allowing the plate to incubate at room temperature for an additional 5 minutes.

Once the plate had been allowed to incubate, 30 μL of substrate was added to each well except well #A1. The plate was then placed on a plate shaker for 5 minutes before allowing the plate to incubate at room temperature for 25 minutes. The absorbance was then immediately read at a wavelength of 410 nm.

Using the assay described above, the IC₅₀ of Glu-boroAla at pH 8 was determined to be 72 nM, the IC₅₀ of Glu-boroPro was determined to be 2.4 μM, and the IC₅₀ of the Glu-boroEtg was determined to be 49 nM.

### Example 2

### Synthesis of L-Ala-[5-(HOCH₂)-2-boroPro] (I)

The synthesis of L-Ala-[5-(HOCH₂)-2-boroPro] is illustrated in Scheme 1.

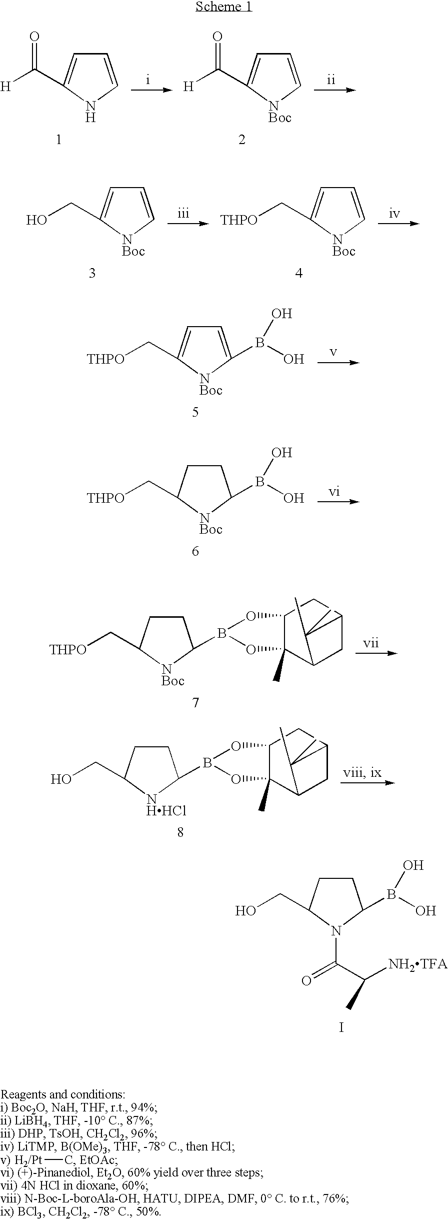

Starting from commercially available pyrrole-2-carbaldehyde 1, synthesis of L-Ala-[5-(HOCH₂)-2-boroPro](I) was achieved via nine steps in an overall yield of 17%. First, pyrrole-2-carbaldehyde 1 was deprotonated with sodium hydride in tetrahydrofuran and then reacted with di-tert-butyl dicarbonate to give N-Boc-pyrrole-2-carbaldehyde 2 (see Tietze, et al. Synthesis of N-protected 2-hydroxymethylpyrroles and transformation into acyclic oligomers. Synthesis (1996), 7:851-857). Reduction of the carbaldehyde 2 with lithium borohydride at −10° C. yielded the hydroxymethyl compound 3. The hydroxylmethyl group of compound 3 was then protected with a tetrahydropyranyl group to form the THP ether 4. Total yield of the first three steps was 78%, with purification by silica gel flash chromatography at each step. The protected pyrrole was deprotonated with LiTMP (generated from n-butyl lithium and tetramethylpiperidine in THF at −78° C.) (see Kelly, et al. The efficient synthesis and simple resolution of a prolineboronate ester suitable for enzyme-inhibition studies. Tetrahedron (1993), 49(5): 1009-16) and quenched with trimethyl borate, then HCl was added to hydrolyze the dimethyl boronate to give the boronic acid 5. Without further purification, compound 5 was hydrogenated over 5% Pt/C in ethyl acetate to afford pyrrolidine-2-boronic acid 6. Crude 6 was stirred with 1.05 eq. (+)-pinanediol in ether at room temperature and then purified by silica gel flash chromatography to yield the protected 5-hydroxymethylboroPro pinanediol ester 7 in 60% yield over these three steps. Removal of the tert-butoxycarbonyl (Boc) group with 4 N HCl in dioxane gave intermediate compound 8 in a yield of 94%. Compound 8 was coupled with N-Boc-L-Ala-OH in the presence of HATU and DIPEA, then the Boc and pinane protecting groups were deprotected with BCl₃ to give the target dipeptide boronate I in a 38% yield over the last two steps.

### Example 3

### Synthesis of L-Ala-5-Me-boroPro (II)

The synthesis of L-Ala-5-Me-boroPro is illustrated in Scheme 2:

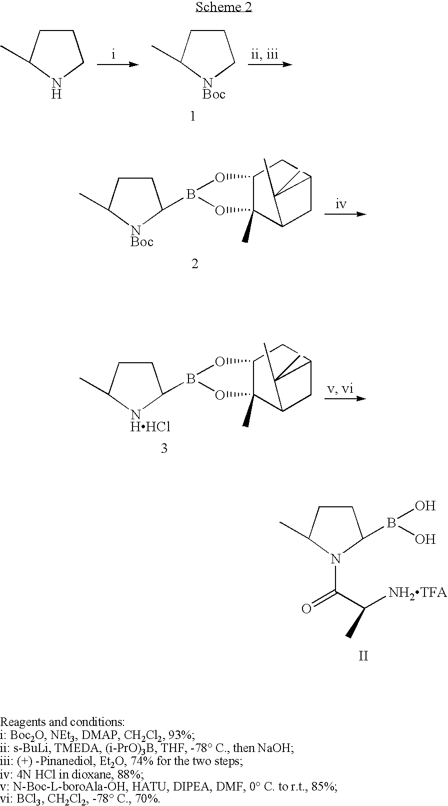

L-Ala-5-Me-boroPro(II) was synthesized from commercially available 2-methylpyrrolidine, as shown in Scheme 2. First, 2-methylpyrrolidine was reacted with di-tert-butyl dicarbonate in the presence of triethylamine and DMAP to give N-Boc-pyrrolidine 1. The C-lithiation of N-Boc-pyrrolidine was achieved using s-BuLi (2.2 equiv.) in THF-TMEDA (see Gibson, et al. A Practical Synthesis of L-Valyl-pyrrolidine-(2R)-boronic Acid: Efficient Recycling of the Costly Chiral Auxiliary (+)-Pinanediol. Organic Process Research & Development (2002), 6(6): 814-816.) at −78° C. and then quenched by triisopropyl borate. After workup with NaOH and then HCl, the crude boronic acid was protected with (+)-pinanediol. The pure boronate compound 2 was then obtained in a yield of 51% over two steps after purification with silica gel flash chromatography. Removal of the tert-butoxycarbonyl (Boc) group with 4 N HCl in dioxane gave the intermediates 5-methylboroPro pinanediol ester 3. Compound 3 was coupled with N-Boc-L-Ala-OH in the presence of HATU and DIPEA, then the Boc and pinane protecting groups were removed with BCl₃ to give the target dipeptide boronate II in a 60% yield over the last two steps.

### Example 4

### Synthesis of L-Ala-cis-boroHyp (III) and Ala-trans-boroHyp (IV)

The syntheses of L-Ala-cis-boroHyp and Ala-trans-boroHyp are illustrated in Scheme 3.

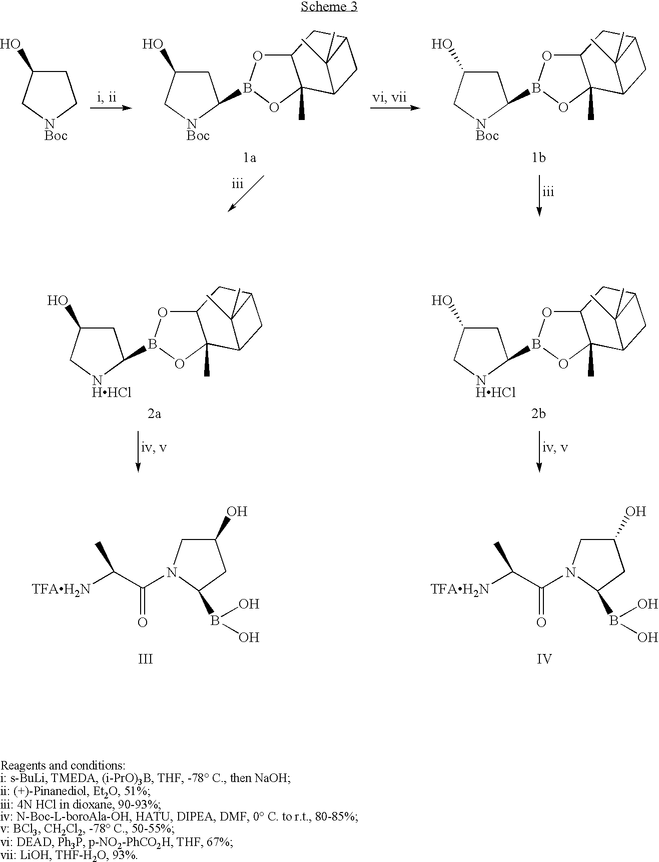

L-Ala-cis-boroHyp (III) and L-Ala-trans-boroHyp (IV) were synthesized from commercially available N-(tert-Butoxycarbonyl)-(S)-(+)-3-pyrrolidinol, as shown in Scheme 3. First, C-lithiation of N-Boc-3-hydroxypyrrolidine was conducted using s-BuLi (2.2 equiv.) in THF-TMEDA (see Gibson, et al., cited above) and the reaction was quenched by triisopropyl borate. After workup with NaOH and then HCl, the cis-2,4-disubstituted adduct was afforded as the major diastereomer. The boronic acid was protected with (+)-pinanediol and then crystallized from ethyl acetate to give the pure boronate compound 1a in a yield of 51% over two steps. The 4(R)-boroHyp derivative 1b was obtained by inverting the configuration at the C-4 atom from 1a via the Mitsunobu reaction (see Hodges, et al. Stereoelectronic Effects on Collagen Stability: The Dichotomy of 4-Fluoroproline Diastereomers. J. Am. Chem. Soc. (2003), 125(31): 9262-3) in a 62% yield. Removal of the tert-butoxycarbonyl (Boc) group in 1a or 1b with 4 N HCl in dioxane gave cis-boroHyp pinanediol ester 2a or trans-boroHyp pinanediol ester 2b. Compound 2a or 2b was coupled with N-Boc-L-Ala-OH in the presence of HATU and DIPEA, then the Boc and pinane protecting groups were removed with BCl₃ to give the target dipeptide boronate III or IV in a 40-45% yield over the last two steps.

### Example 5

### DPIV Inhibition Assays

The compounds prepared in Examples 2-4 were tested in the assay described in Example 1.

L-Ala-[5-(HOCH₂)-2-boroPro] was found to have an IC₅₀ of 21.92 nM at pH 2 and an IC₅₀ of 12.88 μM at pH 8.

L-Ala-5-Me-boroPro was found to have an IC₅₀ of 11.04 nM at pH 2 and an IC₅₀ of 15.41 μM at pH 8.

L-Ala-cis-boroHyp was found to have an IC₅₀ of 2.95 nM at pH 2 and an IC₅₀ of 5.44 μM at pH 8.

L-Ala-trans-boroHyp was found to have an IC₅₀ of 31.13 nM at pH 2 and an IC₅₀ of 64.29 μM at pH 8.

Based upon these data, it was determined that for hydroxylated boroPro-type inhibitors, the hydroxyl group is preferably cis to the boronic acid moiety (or its precursor). Moreover, based upon these results, one of ordinary skill in the art could modify the compounds disclosed in U.S. Pat. No. 6,803,357; U.S. application Ser. Nos. 10/496,706 and 10/496,627, each filed May 25, 2004; and U.S. Provisional Application No. 60/584,581, filed Jul. 1, 2004, the contents of which are incorporated herein by reference in their entirety, by the addition of a hydroxyl group on the ring of a boronic acid-modified proline, preferably on the 4-position of the ring and/or preferably cis to the boronic acid group (or its precursor).

L-Ala-[5-(HOCH₂)-2-boroPro] was also tested to determine its inhibition of dipeptidyl peptidases 8 and 9 (DP8 and DP9). The assay is the same as that described in Example 1, except that DP8 or DP9 was substituted for DPIV. At the pH values tested, L-Ala-[5-(HOCH₂)-2-boroPro] was found to have an IC₅₀ in excess of 70 μM.

### Example 6

### Selectivity for Dipeptidyl Peptidase Isoforms

The assay described in Example 1 was used to determine the IC₅₀ values for several compounds of the invention. In this example, the assay was conducted for DPIV and DP9. The ratio of IC₅₀ values for each tested compound was calculated in order to determine the selectivity for the DPIV isoform. IC₅₀ values were measured at the same pH throughout the assay.

| | DPIV | DP9 | DP9/DPIV |
|---|---|---|---|
| Compound | IC₅₀ (nM) | IC₅₀ (nM) | ratio |
| L-Ala-[5-(HOCH₂)-2-boroPro] | 40 | 2.80 × 10⁷ | 700,000 |
| Arg-boroPro | 2 | 824 | 412 |
| Glu-boroPro | 3 | 20 | 6.67 |
| Asp-boroAla | 796800 | 5 × 10⁶ | 6.28 |
| Arg-boroAla | 8 | 23 | 2.88 |
| Arg-boroEtGly | 10 | 7 | 0.70 |

Although all compounds except Arg-boroEtGly show a degree of selectivity for DPIV over DP9 (and presumably over the similar DP8), the 5-hydroxymethylated boroPro compound is highly selective for DPIV. Based upon these data, it is expected that addition of hydroxy-, alkoxy-, alkyl-, or hydroxyalkyl-containing moieties to a boronic acid-modified proline will result in greater selectivity of an inhibitor for DPIV. Moreover, such a group is preferably cis to the boronic acid group (or its precursor) of boroPro. Accordingly, preferred compounds of the invention inhibit DPIV at least 10 times, preferably at least 100 times, more strongly than they inhibit DP8 and/or DP9, i.e., have an IC₅₀ at least 10 (or 100) times lower against DPIV than against DP8 and/or DP9.

Based upon these results, one of ordinary skill in the art could modify the compounds disclosed in U.S. Pat. No. 6,803,357; U.S. application Ser. Nos. 10/496,706 and 10/496,627, each filed May 25, 2004; and U.S. Provisional Application No. 60/584,581, filed Jul. 1, 2004, the contents of which are incorporated herein by reference in their entirety, by the addition of hydroxy-, alkoxy-, alkyl-, or hydroxyalkyl-containing moieties to a boronic acid-modified proline, preferably cis to the boronic acid group (or its precursor) of boroPro and/or preferably in the 5-position for alkoxy-, alkyl-, or hydroxyalkyl-containing moieties or in the 4-position for hydroxyl moieties, in order to obtain an inhibitor with greater selectivity for DPIV.

### IV. Equivalents

Those skilled in the art will recognize, or be able to ascertain using no more than routine experimentation, many equivalents to the specific embodiments of the invention described herein. Such equivalents are intended to be encompassed by the following claims.

All of the above-cited references and publications are hereby incorporated by reference.

1. A compound having a structure of Formula I: 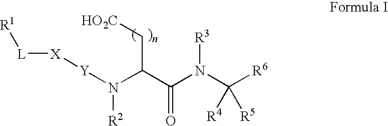
or a pharmaceutically acceptable salt thereof, wherein:
R¹ is selected from the group consisting of H, alkyl, alkoxy, alkenyl, alkynyl, amino, alkylamino, acylamino, cyano, sulfonylamino, acyloxy, aryl, cycloalkyl, heterocyclyl, heteroaryl, and a polypeptide chain of 1 to 8 amino acid residues;
R² is H, lower alkyl, or aralkyl;
R³ and R⁴ are independently selected from the group consisting of H, halogen, and alkyl, or R³ and R⁴ taken together with the atoms to which they are attached form a 3- to 6-membered heterocyclic ring;
R⁵ is H, halogen, lower alkyl, or aralkyl;
R⁶ is a functional group that reacts with an active site residue of a targeted protease to form a covalent adduct;
R⁷ is selected from the group consisting of H, aryl, alkyl, aralkyl, cycloalkyl, heterocyclyl, heteroaryl, heteroaralkyl, and polypeptide chains of 1 to 8 amino acid residues;
L is absent or selected from the group consisting of alkyl, alkenyl, alkynyl, —(CH₂)ₘO(CH₂)ₘ—, —(CH₂)ₘNR²(CH₂)ₘ— and —(CH₂)ₘS(CH₂)ₘ—;
X is absent or —N(R⁷)—, —O—, or —S—;
Y is absent or —C(═O)—, —C(═S)—, or —SO₂—;
m is, independently for each occurrence, an integer from 0 to 10; and
n is an integer from 2 to 6.

2-4. (canceled)

5. A pharmaceutical composition comprising a pharmaceutically acceptable carrier; and a compound of claim 1.

6-8. (canceled)

9. The compound of claim 1, wherein R¹ represents H or lower alkyl, R³ is H, and R⁴ is lower alkyl, or R³ and R⁴ taken together with the carbon to which they are attached form a 5-membered ring, and n is 2.

10. The compound of claim 1, wherein R¹ represents H or lower alkyl, R³ represents H, R⁴ represents H or lower alkyl, R⁵ represents H, and n is 2.

11. The compound of claim 1, wherein L, X, and Y are absent, R¹ is a polypeptide chain of 2 to 8 amino acid residues, and a proline residue is directly attached to the nitrogen substituted with R².

12. The compound claim 1, wherein R¹ is a polypeptide chain of two amino acids, and a proline residue is directly attached to the nitrogen substituted with R².

13. The compound of claim 1, wherein R⁶ is a functional group selected from the group consisting of boronic acid, boronic ester, —CN, —SO₂Z¹, —P(═O)Z¹, —P(═R⁸)R⁹R¹⁰, —C(═NH)NH₂, —CH═NR¹¹, and C(O)—R¹¹
wherein
R⁸ is O or S;
R⁹ is N₃, SH₂, NH₂, NO₂, or OLR¹², and
R¹⁰ is lower alkyl, amino, OLR¹², or a pharmaceutically acceptable salt thereof, or R⁹ and R¹⁰ taken together with the phosphorus to which they are attached form a 5- to 8-membered heterocyclic ring;
R¹¹ is selected from the group consisting of H, alkyl, alkenyl, alkynyl, —NH₂, —(CH₂)_{q}—R¹², —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R¹², —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R¹², —C(O)NH₂, —C(O)OR¹³, and -(Z¹)(Z²)(Z³);
R¹² is H, alkyl, alkenyl, aryl, heteroaryl, cycloalkyl, cyclcoalkenyl, or heterocyclyl;
R¹³ is H, alkyl, alkenyl, or LR¹²;
Z¹ is halogen;
Z² and Z³ are each independently H or halogen;
p is, independently for each occurrence, an integer from 0 to 8; and
q is, independently for each occurrence, an integer from 1 to 8.

14. The compound of claim 1, wherein R⁶ is boronic acid.

15. The compound of claim 1, wherein R¹ is H; R² is H; R³ is H; R⁴ is H or lower alkyl; R⁵ is H or lower alkyl; and R⁶ is boronic acid.

16. The compound of claim 1, wherein L, X, and Y are absent.

17. The compound of claim 1, wherein L, X, and Y are absent; R¹ is H; R² is H; R³ is H; R⁴ is CH₃; R⁵ is H; and R⁶ is boronic acid.

18. The compound of claim 1, wherein L, X, and Y are absent; R¹ is H; R² is H; R³ is H; R⁴ is CH₃; R⁵ is H; R⁶ is boronic acid; and n is 2.

19. The compound of claim 1, wherein said compound is represented by:

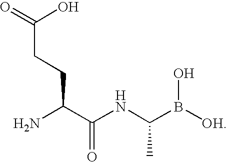

20. A method of lowering blood glucose in a subject with type 2 diabetes, comprising the step of administering to a subject in need thereof a therapeutically effective amount of a compound of any one of claims 1-19.
